SEQUENCE LISTING (20 sequences)

SEQ ID NO: 1 - DNA, 481 bp, Homo sapiens
atggcccgaaccaagcagactgcgcgcaagtcaacgggtggcaaggcgccgcgcaagcagctggccaccaaggtggctcgcaagagcgcacctgccactggcggcgtgaagaagccgcaccgctaccggcccggcacggtggcgcttcgcgagatccgccgctaccagaagtccactgagctgctaatccgcaagttgcccttccagcggctgatgcgcgagatcgctcaggactttaagaccgacctgcgcttccagagctcggccgtgatggcgctgcaggaggcgtgcgagtcttacctggtggggctgtttgaggacaccaacctgtgtgtcatccatgccaaacgggtcaccatcatgcctaaggacatccagctggcacgccgtatccgcggggagcgggcctaggagggctatctcgccacctgagaggttgcgcaacgttcaccccaaaggctcttttaagagccacccacct

SEQ ID NO: 2 - protein, 136 aa, Homo sapiens
MARTKQTARKSTGGKAPRKQLATKVARKSAPATGGVKKPHRYRPGTVALREIRRYQKSTELLIRKLPFQRLMREIAQDFKTDLRFQSSAVMALQEACESYLVGLFEDTNLCVIHAKRVTIMPKDIQLARRIRGERA

SEQ ID NO: 3 - DNA, 1499 bp, Homo sapiens
acttccgtccggtctgcctggtctctctaaccgcgccagtgtgcctccgactcggaacggcttccgcggccggggcagcgagggccgggggcggcgggcgggatgagtgcggtgtgcggtggagcggcgcggatgctgcggacgccgggacgccacggctacgccgccgagttctccccgtacctgccgggccgcctggcctgcgccaccgcgcagcactacggcatcgcgggctgtggaaccctactaatattggatccagatgaagctgggctaaggctttttagaagctttgactggaatgatggtttgtttgatgtgacttggagtgagaacaacgaacatgtcctcatcacctgtagtggcgatggctcgctgcagctctgggacactgccaaagctgcagggccactgcaagtctataaagaacacgctcaggaggtgtatagtgttgattggagccaaaccagaggtgaacagcttgtggtgtctggctcatgggatcaaactgtcaaattgtgggatccaactgttggaaagtctctgtgcacctttagaggccatgaaagtattatttatagcacaatctggtctccccacatccctggttgttttgcttcagcctcaggtgatcagactctgagaatatgggatgtgaaggcagcaggagtaagaatcgtgattcctgcacatcaggcagaaatcttgagttgtgactggtgtaaatacaatgagaatttgctggtgaccggggcggttgactgtagtttgagaggctgggacttaaggaatgtacgacaaccagtgtttgaacttcttggtcatacctatgctattaggagggtgaaattttcaccatttcatgcttctgtgctggcctcttgctcgtatgattttactgtaagattctggaacttttcaaagcctgactctcttcttgaaacagtggagcatcatacagagtttacttgtggtttagacttcagtcttcagagccccactcaggtggctgactgttcttgggatgaaacaataaagatctatgaccctgcttgtcttactattcctgcttgagatacactactttggtcagaaacagaggatgttggctgaagaactgcctaacagcaaataaattaactatggaaaacatagacattatgcttttatatgctattcagatttcaaatctttccaatttaccctggaatcagttttgagggagctgataaagactttagctgactcgttaagcctgatacataagccatatttaaaattctaagaaataattaatgttatgatatatcttgtagtatctattaaaatgtctctgggtcataaaatggattaaaatatgggagatcagtaggttatacttatatagatagtgatatatttcatttttaatttgtcatttttgatgtaaaatataatcactgctgtgataaataaactatctattgatcatttatcattttaaaaaaaaaaaaaaaaaa

SEQ ID NO: 4 - protein, 323 aa, Homo sapiens
MSAVCGGAARMLRTPGRHGYAAEFSPYLPGRLACATAQHYGIAGCGTLLILDPDEAGLRLFRSFDWNDGLFDVTWSENNEHVLITCSGDGSLQLWDTAKAAGPLQVYKEHAQEVYSVDWSQTRGEQLVVSGSWDQTVKLWDPTVGKSLCTFRGHESIIYSTIWSPHIPGCFASASGDQTLRIWDVKAAGVRIVIPAHQAEILSCDWCKYNENLLVTGAVDCSLRGWDLRNVRQPVFELLGHTYAIRRVKFSPFHASVLASCSYDFTVRFWNFSKPDSLLETVEHHTEFTCGLDFSLQSPTQVADCSWDETIKIYDPACLTIPA

SEQ ID NO: 5 - protein, 323 aa, Homo sapiens
MSAVCGGAARMLRTPGRHGYAAEFSPYLPGRLACATAQHYGIAGCGTLLILDPDEAGLRLFRSFDWNDGLFDVTCSENNEHVLITCSGDGSLQLWDTAKAAGPLQVYKEHAQEVYSVDWSQTRGEQLVVSGSWDQTVKLWDPTVGKSLCTFRGHESIIYSTIWSPHIPGCFASASGDQTLRIWDVKAAGVRIVIPAHQAEILSCDWCKYNENLLVTGAVDCSLRGWDLRNVRQPVFELLGHTYAIRRVKFSPFHASVLASCSYDFTVRFWNFSKPDSLLETVEHHTEFTCGLDFSLQSPTQVADCSWDETIKIYDPACLTIPA

SEQ ID NO: 6 - protein, 136 aa, Homo sapiens
MARTKQTARKSTGGKAPRKQLATKVARKSAPATGGVKKPHRYRPGTVALREIRHYQKSTELLIRKLPFQRLMREIAQDFKTDLRFQSSAVMALQEACESYLVGLFEDTNLCVIHAKRVTIMPKDIQLARRIRGERA

SEQ ID NO: 7 - protein, 136 aa, Homo sapiens
MARTKQTARKSTGGKAPRKQLATKVARKSAPATGGVKKPHRYRPGTVALREIRRYQKSTELLIRKLPFQRLMREIAQDFKTDLRFQSSAVMALQEACESYLVGLFEDTNLCVIHAKRVTIMPKDIQLACRIRGERA

SEQ ID NO: 8 - protein, 136 aa, Homo sapiens
MARTKQTARKSTGGKAPRKQLATKVARKSAPATGGVKKPHRYRPGTVALREIRRYQKSTELLIRKLPFQRLMREIAQDFKTDLRFQSSAVMALQEACESYLVGLFEDTNLCVIHAKRVTIMPKDIQLARCIRGERA

SEQ ID NO: 9 - DNA, 12901 bp, Homo sapiens
cattatctacatttctttttttttttttttttttctgagaggtagcctcgttttgtcacccaggctggagtacagtggcttgatcttggctcactgcaacctccacctcctgggttcaagcaattctcctgcctcagcctcccgagtagctgggattacaggcatgcgccaccacgctcagctaatatttttgtatttttagtagagattgggtttcactatgttggtcaggctggtctcgaactcctgaacttaaatgatccacccgccccagcctcccaaagtgctggcattacagacagatgtaagccactgcacccagcctacgttttctacatctctttttttttttttttgagaccgtgtcttgctctgtcgcgcccaggctggagtggaatggcgcgatctcagctcactgcaagctccgcctcctgggttcacgccattctcctgcctcagcctcccgagtagctgggactacaggtgcccgccaccaagcccggctaattttttgtatttttagtagagacggggtttcactgtgttagccaggatggtctcaatctcctgaccttgtgatccgcccgcctcggcctccgaaagtgctgggattacaggcttgagccaccgtgcccggccacgttttctacatttcttagtccacttggtgaactcttcctccttcaggcctcatatgtacattttctgcctcttccagggttagagcgctgctctctagcagcatcttggacgtgtggcaagatctgcagtcgtggtttgtttccaggacctactcatttagactgtgagcacctttaggaaggcagaattgtctttgggccctagtgatacttgttgaaattgttagtttagtagaagagtacattgaaaggggaagaggcaggtggatcacctaaggtcaggaatttgagagcatcctggccaacatggtgaaattctgtctctactaaaagtacaaaacaattagctgggcgtggtggcacgtacctgtagtcccggctactagggaggctgaggcaggagaatcgcttgaacctgggaggtggaggttgcagtgagctgagatcacgctactgcactccagcctgggcaaaagagcaagatttcctctcaaaaaaaaaaaaaaaagaaaaggtggggagaaggaagaggtggcacctttgatctttgagcttttcaggcccctgggcctgggtctgacttgttgtaatgggttgtatgtgttctagttgcaggaaatcacgtttaagaattactacacagcttttttgagcatccgtgtccgtcagtacacctcagcacacacacctgccaagtgggtgacctgcctgcgggactactgcctaatgcctgacccacacagtgaggagggagcccaggagtatgtatcgctgttcaagcatcaggtcagctgggcctcaggatggccagggcagcccagatgaggacttaccagaggtggggggattggatcaatataccaagagggaagagggggccagtctggcctgaactgtgacttccagggaagtgccttggagggactactgttccttccttcccaaggcctgtggggtgggcagcattctggctcattgctggttgggaggcttctgggcagtgagctggatccttgctgggtccttgtgtgtgtgtgtcagatgctgtgtgacatggctagaatatcggagctacgcctgattctgcggcagccatcaccactgtggctgtctttcacagtggaggagctgcagatctatcagcagggaccaaaggtaagtgactagctcagctgctggctggccctttccccagaaagcttctatgactgggaaagctaggtggtggggtgggtaatgggtgaagattgtgaatggggccttcatggaggttagctgggtgtgaatgctggaagctcacaatgagaggtgaggccatgtccccatatcccctgacctttggggccacctgctactttcagagctggagcctttaggtgctgggagacacttttgagcttgtgtttctgaggactccgtctgcacagcaggagaacttaggtagtctgactgcctctttaggtctctgtgggctccctaaacagggttttcccagtgtctgaggcccagcggcccttctctctgctcctaggaccttggaatctggtcccagagctgtccactttgtctcactcagggcagtacatctttgctttgcagagcccctccgtgacctttcccaagtggctctcccacccagtgccctgtgagcaacctgcactcctccgtgaggtaagccctcaccctaggtaagaaccatgtgtgaatggctctagtgtccaacacaggccaggtagaccccttcctgtaaaattctggggctgggcccagggcccttgggtgttatcttcctcctgggtattctgtgggatttgaggcatctctgttgtggggctacatcaggccctaacctcatgtttcataagccatctgagaatgttgttggtctcagctggagggcccctagcccaggttggcctctctggagcctgaagggcactgctggggagagacccatggccactgacccctccttctggagcagggtctcccagaccccagcagggtatcctccgaggtgcagcagatgtgggcactgacagagatgatccgggccagtcacacctccgcaaggatcggccgctttgatgtaagtaatcgcttccttctgcttggcttgctttgccctgagcagatcagacctgatggtcttttgtatttcaggtggatggctgttatgacctgaacttgctctcctacacttgaatggttgctcctagccaagatgttggcctttctgtgcccacccagacttggtttgggccaccagaggctgaagtgtatttcctgtgcattccaagtgttgcctgcatgcctgttctgtctgggccacattcacagattcaggattctcaggggcttaccgtgttacttcagcatgagctcctggcatgggggtgctctgtcttcgcctggctgcaactcaattcttccttctacccaaaaacactccagtcagttgtacttactgagaatccctgagtgccctctgccccttctggcaacatgcacattcctgtcttctgcctgctccctcatgtcagtgcagttgggctgggctgacctactaaaacctgctccatgtatggacctggaaggccattgctgccatggcacttgggacacttgaggaagaaacagaaaccttatttcttcctgtgtgtcccctagtggttttctttccatttgtgttatgccacagagagaaatttcctgtctgaactcttctgctgcctgcctaggaaggaaacctgaagatttgggcagcctgagtctcttggtttctgagctggctcctgaatatatgttcaagtgggtgtgcccatagggcctgggcacgtgtctctagcaaaaccagggcctgagcataagaactgggacccttctaggcagggtgcagtggctcacgcctgtaatcccagcactttgggaggcccaggcaggcggatcacctgaggtcaggagttcaagaccagcctgaccaacctggagaaaccccgtctctactaaaaatacaaaaaattagctgggcgtggtggtaggcacctgtaatcccagctacttgggaggctgaggcaggagaatctcttgaacccgggaagtggaggttgcggacctgagatcatgccattgcactccagcctgggcaagaagagcgaaactccatcttaaacaaacaaacaaaaaaaaaagaactgggacccttctgccatctgacatagcccaaagcacatctctatcctttctcccagttgcccctctccttttttgttgttttttttgaggttgagttttgctcttgttgcccaggctggagtgcaatagtgcaatcttggctaactgcaacctccgcctcccaggttcaagcaattctcctgcctcagtctcccgagtagctgggattacagtcatgcatcaccatgcctggctaattttgtatttgtagtagagatggggtttctccatgttggtcaggctggtctcaaacacctgacctcaggtgatctgcctgccttggccttccaaagtgctgggattacaggcatgagccaccgcgcccgccctgcccctttccaagagatatgctcagcataatgagagatcaaccgtggtggggagtgggtccaagtaagggttctcattgttgccttttaagaggcctaagtcctgcattaactcagtccaaccattaagtgatgcaggctgctcttccctcatgtgagaaaaacctgtttccctctggttcttcctggctttgcacctggtggctattacaaagcagtactgcagtatgtcttggacctggaaagttggacatgtgtgtggctcaacagttttctcgggggagttcagtgaccttttggtggccaccctctgttctcacctgtgagttcttgcaggtggcgacttcatctgcaaggtcacttacatagccccactcagctgcttcggaaagcgtgacgaagcagtagtcctcagtgtgtgtgtaagtgaagaggaccttgtggtagcagggaccatgcttcattcgtggctgtgtccccatctgagggcctggtatgcagtaagatcaataaatacttgtggaatgcatgactgctggctgcctgtgtctgcctggtgggagagctgtggaactaggggtccacatgaggacagactactgtggtgctgcctacctgccaggctggcccaccttccccacccccacttgaccttactctcttgaggtcctaaaggtatgattcagacctttcagccctttccagaactttgaccctctggagacagggcggttctgcagggggcggtctagcccctagctttgttctgggcagcccaatcagctctggattgcccgagctgccctgaagagtcagccgaaagagctggaggcttcttccctgtggttgcgggttcctgcattctctgctctaaatcccagcctgcccttggggctgcccacgcccccttcagatcctttgctccggagagagacctgtccgagcagaggcctggactacatctcccggcgtgcctggcagtgtggtggcctctgtgcgccgtctgcactcgttgcaggcgacgatgcagagggctgtaagtgtggtggcccgtctgggctttcgcctgcaggcattccccccggccttgtgtcgtccacttagttgcgcacaggtgggatagagggtgctgatcttggagggtgggggagggaagcggctgggggctagggccctggtggcactaagcccagccgcccacaggaggtgctccgcaggacaccgctctatgacttccacctggcccacggcgggaaaatggtggcgtttgcgggttggagtctgccagtgcagtaccgggacagtcacactgactcgcacctgcacacacgccagcactgctcgctctttgacgtgtctcatatgctgcaggtgagccaggggagcacactggccatctgatcaggccttcctccctgtttacagtggtccactccttggctttgtttgctgtgaactggattccaaggacagccttctcagaagagctagaggtcatgggggtgtcatggataggtcaatggccccaaggaaatgagctcagagggtctcatcatctcatccttctagcttgcccccattaaatggtgtgccttaatgtcccgggttttccagggctgacacccactctcatgtattgggctggatgttggtttgaaaaggggttatttcaggaaggtgctaggtcctgtccagttccctggcagtccccccacacagagtgactgggggaggtcaggaaatcagaaggtggtcctttgccacgtgtagaaccaaaagactggttagtcactttgggtgggctggactgcactgtggatggcagggaggatgcatccttgcttagtagccttttggattgtagctgttgcaatggcctgaggctttcaatctcttccccagaccaagatacttggtagtgaccgggtgaagctgatggagagtctagtggttggagacattgcagagctaagaccaaaccaggtgggcccttttatttctgctctatgagttttcttagtcatgagccagcaaatgggctgagtccctacctcaggcttaggggcacagagatgagtcaaacacagcttctcagggcttgcagtcttggacagaatccctgagtcatttccagagacacagacatttgtagggaggggaatatcccagtcttactacatcaggctaggaggccagaaaccaaggttaatatgctgctctctggccatcccttctgcttaacccctctctattcaacatgtggtccctggccaggcgacgtgactcaaacacctgtaatcctggcacttggggaggccaaggcgggtggatcacctgaggtcaggagttcgagaccagcctggccaacatggcaaaacctcgtttctactaaaaacacaaaaattagccgggcgtggtggcgcgtgcttgtagtcccagctactcgggaggctgaggttgcagcaagctgaggttgcaccactgcactctggcctgggcaacagagtgagactcagtctaaaaagaaaaataacaacaacaaagtgtggtctctaaaccagcatctttggagcttgttaggaatatagtctcaggcactgctgcagacctgctgaatcagaaccttcattttaacaagatccccaggtgattcatgtgaccccctcaacctcaggctttttgtccatccagtggggaggctaaggaagttttttgggggcattgctatggagtacttaggagagcctgcgatcatcaagatgacctttgaagcttttgtcctgtgggttgccacatgggctaagtcgtcccaggtttgggtaggtgaagcagcaggtcctaggtgtcatagattaagtgtcagatgggactctcagtacagagtggggaggggtgcagaggttgcaccagatcagggaagcaccccaaggtagggaggggatgaaaacttacttgatgcatgaggatcaaagaccttggacttgggcccagacttgtaccttgcacaccctgagtccttcacattagggttcataaccttttggtaactaagtgggtccagggttgtgtctggctcacccttggccttgccctcctggacctggaagccaaaacgctccactttggtcctaggggacactgtcgctgtttaccaacgaggctggaggcatcttagatgacttgattgtaaccaatacttctgagggccacctgtatgtggtgtccaacgctggctgctgggagaaagatttggccctcatgcaggtataccccctctgtgttctagacaccttgtcttccttgttcatagagcagtatccttgttgtccaagacagggttggcacagacaagggacctggaaaatggctctttcagtgaatgaacagctatgagactcaagagcaggccctctctggagcacgccagggtttggtggtgggggattaccaggcatggggagggggtcttggcccctctggtgtgaggtggcagaactgggcttgggtcatctctatcacttagtagctatgtgaccttgtaagatgggaaccagcctactacctttttttttcctccctcctcctcctcctctccactgtcctactactgtccagctctgcagcgtccagcccatggtagctgctcagtaagtggccccagatgctcttgtgtttctgtcttttaggacaaggtcagggagcttcagaaccagggcagagatgtgggcctggaggtgttggataatgccctgctagctctgcaaggtgagagggctgggctggggttggagccaacctttgccttcctggcttcattgactgcttagcacagaggccataccaagaagaatgtgtggaaggtgtgatatttgtgtgccataattttaagttttgtatcttgccctcctcctacctaccacaaggctccatgggctagggacaagggcatcttctcccacttgcagagtaagacatgctcagggcttgttctggtccctgaataaggctttagtccagctttgagggatgctgggacctggatacttggtcactggctccctgggcccaggccccactgcagcccaggtactacaggccggcgtggcagatgacctgaggaaactgcccttcatgaccagtgctgtgatggaggtgtttggcgtgtctggctgccgcgtgacccgctgtggctacacaggagaggatggtgtggaggtgtgtcaagaagtggtgtgggggcagtacagggcacaggatgcatgtgaggggttgggggtatgtggtacagggagcttcccatctgccctctggtgtttcatacagatctcggtgccggtagcgggggcagttcacctggcaacagctattctgaaaaacccagaggtgaagctggcagggctggcagccagggacagcctgcgcctggaggcaggcctctgcctgtatgggaatgacattgatgaacacactacacctgtggagggcagcctcagttggacactgggtgagctgggccagcactaaaggatagggtcctggaggtcagggtgacccttgataagactagccagcccatgactcatccaaaggttatgtagcctgaagccttcctgagcctcccttccctgaaggtgccactgtgcctttgcccattagaaactgcagatgttggccggatgcggtggctcacatctgtaatcccagcactttgggaggccgaggcgggtggatcacgaggtcaggagatcaagaccatcctggctaacatggtgaaaccccgtctctactaaaaatacaaaaaaaaaattagctgggcattgtggcgggcgcctgtagtcccagctactcgggaggctgaggcaggagaatggcatgaacctgggcggcggagtttgcagtgagccgagattgcgccactgcacctccagccagggcgatagagcgagaccccgtctcaaaaaaaaaaaaaaaaaaaaaaaagaaactgaagatgctgccgggcatggtggctcacacctgtaatcccagcactttgggaggccaaggtgggtggatcatgaggtcaggaattcgagaatagcctggccaacatggtgagaccccgtctctactgaaaatataaaaattagccaggcgtggtggcgggtgcctgttatcccagctactcgggagactgaggcaggtaaattgcttgaacctgggaggcagagtttgcagtgagctgagatcgtgccactgcattctaggcctgggcgacagagtgactccatctcaaaaaaaaaaaaaagaaactgaagacgctcggcaggtgggcagcatggcctccaggggataggaggtggttggttggctgacggcagtgaagggaggaatagagcctggagtagcccaagcaaaggggccttgatgctagatagtgtgttacttgaggacctcataaggacctcctgtggccagggcttctatgggctgtggcttatgtctcatgtgtcattctccagggaagcgccgccgagctgctatggacttccctggagccaaggtcattgttccccagctgaagggcagggtgcagcggaggcgtgtggggttgatgtgtgagggggcccccatgcgggcacacagtcccatcctgaacatggagggtaccaagattggtaggtggaccagggaagctgggaaacccttgtctcttcccaggagggtgggggcactggcagggtggtgctgatgcgtggcttatgcttgcttgacaggtactgtgactagtggctgcccctccccctctctgaagaagaatgtggcgatgggttatgtgccctgcgagtacagtcgtccagggacaatgctgctggtagaggtgcggcggaagcagcagatggctgtagtcagcaagatgccctttgtgcccacaaactactataccctcaagtgaagctggctcagggtggggctgtcccttccaggagttttgcccctacaaggggttagtcaagaagctgaggcagaactcactgggggtgggcagttaaggtggaggctgattctaattgtctggttgaggggccacaccacctattccccccacctaactcatgccattccagcttccttcaggaccctgcttctgagtgacggaccagctcacacaatgtcttgtttcagtccatgatcccactgacctactcttgcctgctggagggtaatgagaagctttggttctgccatctctcccactctgccaggtgctggctgtggagcaaaggctcacctttgtggagaggataaaacctgcccaacctacctcaccatggtttttcacattgcaaagggtaataacatgggcagtgcggacttaggctaccccctccagtttgctttccgtaaatgcaaattgtccttactgcaagtcaggaatgattgctgactcacagtagggctgctatgcctgtgtgtaaacttggggatggctgagggaacatagactcactcttccacattcccaagttggtctagtgtgctgcccagtagcaaaccatggcagactcaccacctattctgagttccagggctgctgtagggcagggtgggcttcctcccagacttgccttaccctgggctgatctttgcccctggtatgcattaatggactccactgaatcctgaaaaaaaaattaaacttccttcttacttgccagtctctagcttcattgttctctgttcacagggttcctgaaatgccaacccaatgcctgccttcctggcctccaagacaagcttggaataggttctccttgaaaggggccagtctataaaaatggagatattgcccctgttgggcaccccatccctgctcctccagggagctgcatcacctctcccctccctgcaagttattgcatctgggtcccaaggggcaacagcttccaggatgttccctctctcactgcccctgtgcatgcacacccttgttctgtctgaataaccacaacaaccgatgcacttccgtgtttaataagccacatcctcagttgagcctggggtgaaatgtgagatcctgactctgtgcagtagtattagtgggtgggccaggggctgtgaataacatcatcctcagtacagctgcaattccagggcccctctaccacaaagatggcttaagcaaaggcagccagatggaagtatgatatccaacaggaaggaagtaggcaggggtcactaaagtggctggtggcccagcagatggaaacagaagtatggccccgagggaaggaggcaggtccagggctacagtgctttcaggtactggtgtttctataggggcatttgccacccacatctttggaaactcccctggcctattgtgacatggcagggctgcctggttcttgaaggtagagaaaatgctagtggggaggagctgagcctgtgacgtgcatttgtaccaggtccagatgggaaatgatgaggctgcaggctatgatggggccactgtgcactaaatgcttggtaccatcctatgaggggacaaggtgcagacccacattcccataggcacttaagcagtggtggataggaggctaccccaggggttcagctgtccttcaggtaatcagcttacccccaagaaggactgagaggaatatgagtaggacagttggcagatgacaatttcctttgcctggctcacgggagaagtctggttgggccaggtatgtgggtgtttggtgcctccatctggatagtgccagcaggtacaccaccctgccttatacctgaatggccagctaaataaccatagagcttaaggtctggctcaggtcaggattccctggctccttgccaacaaagcaaaaccagagattcagaaaactcagggcctaggagaaaacagacctgccttacaaaccccagagctggtcaacctcaggcttcctctcaggaaaaaaattttttttttgagatgggtcttgctctgttgcccaggctggagtacaacggcgcaatcttggctcactgcaacctctgcctcccaggttcaagcaaatctcctgcttcagcctcctgagtagctgggattactggtgtgtgccaccacacccggctaatttttgtatttttagtagagatggggttttcaccatgttggtcaggctggtctcgaactcctgacctcgtgatctgcctgcctcagcctcccaaagtgttgggattacaggcgtgagccaccatgcctggccaactcaggaaaaatttttatggggtcacatcttggggatggaagagggacaggaaaagaaacctaagacaaaagaactgatggtcttcccaacccagaggcttcctgagttatcacccctgagagaggaagaacacaatctctctccttaaggcacccagataagagtacaggcaaactcttcacttgcccagtgcctgggtgagggagcaggcttaagaggcagaagaggcccagccctgtaggcactgggtactccctgctaccaaccctgaacccccggcagttgtccctgaggtctggggagtagcagaacccagtgtagaagccagcagacccagtgatgcccttggagaccctctgctgccttcccttattctctgtgggttttgagaggttcgtttgctgccatttccctgaaagtgatggagagagagaagagagagtggttattccat

SEQ ID NO: 10 - protein, 403 aa, Homo sapiens
MQRAVSVVARLGFRLQAFPPALCRPLSCAQEVLRRTPLYDFHLAHGGKMVAFAGWSLPVQYRDSHTDSHLHTRQHCSLFDVSHMLQTKILGSDRVKLMESLVVGDIAELRPNQGTLSLFTNEAGGILDDLIVTNTSEGHLYVVSNAGCWEKDLALMQDKVRELQNQGRDVGLEVLDNALLALQGPTAAQVLQAGVADDLRKLPFMTSAVMEVFGVSGCRVTRCGYTGEDGVEISVPVAGAVHLATAILKNPEVKLAGLAARDSLRLEAGLCLYGNDIDEHTTPVEGSLSWTLGKRRRAAMDFPGAKVIVPQLKGRVQRRRVGLMCEGAPMRAHSPILNMEGTKIGTVTSGCPSPSLKKNVAMGYVPCEYSRPGTMLLVEVRRKQQMAVVSKMPFVPTNYYTLK

SEQ ID NO: 11 - DNA, 3820 bp, Homo sapiens
ctttgcgcgagtgtcttggttgagcgcagcgcccattcattgcccgcgagcgtccatccatctgtccggccgactgtccagcgaaaggggctccaggccgggcgcagccgccacccgggggaccgaggccaggagaggggccaagagcgcggctgacccttgcgggccggggcaggggacggtggccgcggccatgcagtcctgtgccagggcgtgggggctgcgcctgggccgcggggtcgggggcggccgccgcctggctgggggatcggggccgtgctgggcgccgcggagccgggacagcagcagtggcggcggggacagcgccgcggctggggcctcgcgcctcctggagcgccttctgcccagacacgacgacttcgctcggaggcacatcggccctggggacaaagaccagagagagatgctgcagaccttggggctggcgagcattgatgaattgatcgagaagacggtccctgccaacatccgtttgaaaagacccttgaaaatggaagaccctgtttgtgaaaatgaaatccttgcaactctgcatgccatttcaagcaaaaaccagatctggagatcgtatattggcatgggctattataactgctcagtgccacagacgattttgcggaacttactggagaactcaggatggatcacccagtatactccataccagcctgaggtgtctcaggggaggctggagagtttactcaactaccagaccatggtgtgtgacatcacaggcctggacatggccaatgcatccctgctggatgaggggactgcagccgcagaggcactgcagctgtgctacagacacaacaagaggaggaaatttctcgttgatccccgttgccacccacagacaatagctgttgtccagactcgagccaaatatactggagtcctcactgagctgaagttaccctgtgaaatggacttcagtggaaaagatgtcagtggagtgttgttccagtacccagacacggaggggaaggtggaagactttacggaactcgtggagagagctcatcagagtgggagcctggcctgctgtgctactgaccttttagctttgtgcatcttgaggccacctggagaatttggggtagacatcgccctgggcagctcccagagatttggagtgccactgggctatgggggaccccatgcagcattttttgctgtccgagaaagcttggtgagaatgatgcctggaagaatggtgggggtaacaagagatgccactgggaaagaagtgtatcgtcttgctcttcaaaccagggagcaacacattcggagagacaaggctaccagcaacatctgtacagctcaggccctcttggcgaatatggctgccatgtttgcaatctaccatggttcccatgggctggagcatattgctaggagggtacataatgccactttgattttgtcagaaggtctcaagcgagcagggcatcaactccagcatgacctgttctttgataccttgaagattcagtgtggctgctcagtgaaggaggtcttgggcagggccgctcagcggcagatcaattttcggctttttgaggatggcacacttggtatttctcttgatgaaacagtcaatgaaaaagatctggacgatttgttgtggatctttggttgtgagtcatctgcagaactggttgctgaaagcatgggagaggagtgcagaggtattccagggtctgtgttcaagaggaccagcccgttcctcacccatcaagtgttcaacagctaccactctgaaacaaacattgtccggtacatgaagaaactggaaaataaagacatttcccttgttcacagcatgattccactgggatcctgcaccatgaaactgaacagttcgtctgaactcgcacctatcacatggaaagaatttgcaaacatccacccctttgtgcctctggatcaagctcaaggatatcagcagcttttccgagagcttgagaaggatttgtgtgaactcacaggttatgaccaggtctgtttccagccaaacagcggagcccagggagaatatgctggactggccactatccgagcctacttaaaccagaaaggagaggggcacagaacggtttgcctcattccgaaatcagcacatgggaccaacccagcaagtgcccacatggcaggcatgaagattcagcctgtggaggtggataaatatgggaatatcgatgcagttcacctcaaggccatggtggataagcacaaggagaacctagcagctatcatgattacatacccatccaccaatggggtgtttgaagagaacatcagtgacgtgtgtgacctcatccatcaacatggaggacaggtctacctagacggggcaaatatgaatgctcaggtgggaatctgtcgccctggagacttcgggtctgatgtctcgcacctaaatcttcacaagaccttctgcattccccacggaggaggtggtcctggcatggggcccatcggagtgaagaaacatctcgccccgtttttgcccaatcatcccgtcatttcactaaagcggaatgaggatgcctgtcctgtgggaaccgtcagtgcggccccatggggctccagttccatcttgcccatttcctgggcttatatcaagatgatgggaggcaagggtcttaaacaagccacggaaactgcgatattaaatgccaactacatggccaagcgattagaaacacactacagaattcttttcaggggtgcaagaggttatgtgggtcatgaatttattttggacacgagacccttcaaaaagtctgcaaatattgaggctgtggatgtggccaagagactccaggattatggatttcacgcccctaccatgtcctggcctgtggcagggaccctcatggtggagcccactgagtcggaggacaaggcagagctggacagattctgtgatgccatgatcagcattcggcaggaaattgctgacattgaggagggccgcatcgaccccagggtcaatccgctgaagatgtctccacactccctgacctgcgttacatcttcccactgggaccggccttattccagagaggtggcagcattcccactccccttcgtgaaaccagagaacaaattctggccaacgattgcccggattgatgacatatatggagatcagcacctggtttgtacctgcccacccatggaagtttatgagtctccattttctgaacaaaagagggcgtcttcttagtcctctgtccctaagtttaaaggactgatttgatgcctctccccagagcatttgataagcaagaaagatttcatctcccaccccagcctcaagtaggagttttatatactgtgtatatctctgtaatctctgtcaaggtaaatgtaaatacagtagctggagggagtcgaagctgatggttggaagacggatttgctttggtattctgcttccacatgtgccagttgcctggattgggagccattttgtgttttgcgtagaaagttttaggaactttaacttttaatgtggcaagtttgcagatgtcatagaggctatcctggagacttaatagacatttttttgttccaaaagagtccatgtggactgtgccatctgtgggaaatcccagggcaaatgtttacattttgtataccctgaagaactctttttcctctaatatgcctaatctgtaatcacatttctgagtgttctcctctttttctgtgtgaggtttttttttttttaatctgcatttattagtattctaataaaagcatcttgatcggaagaaaaaaaaaaaaa

SEQ ID NO: 12 - protein, 1020 aa, Homo sapiens
MQSCARAWGLRLGRGVGGGRRLAGGSGPCWAPRSRDSSSGGGDSAAAGASRLLERLLPRHDDFARRHIGPGDKDQREMLQTLGLASIDELIEKTVPANIRLKRPLKMEDPVCENEILATLHAISSKNQIWRSYIGMGYYNCSVPQTILRNLLENSGWITQYTPYQPEVSQGRLESLLNYQTMVCDITGLDMANASLLDEGTAAAEALQLCYRHNKRRKFLVDPRCHPQTIAVVQTRAKYTGVLTELKLPCEMDFSGKDVSGVLFQYPDTEGKVEDFTELVERAHQSGSLACCATDLLALCILRPPGEFGVDIALGSSQRFGVPLGYGGPHAAFFAVRESLVRMMPGRMVGVTRDATGKEVYRLALQTREQHIRRDKATSNICTAQALLANMAAMFAIYHGSHGLEHIARRVHNATLILSEGLKRAGHQLQHDLFFDTLKIQCGCSVKEVLGRAAQRQINFRLFEDGTLGISLDETVNEKDLDDLLWIFGCESSAELVAESMGEECRGIPGSVFKRTSPFLTHQVFNSYHSETNIVRYMKKLENKDISLVHSMIPLGSCTMKLNSSSELAPITWKEFANIHPFVPLDQAQGYQQLFRELEKDLCELTGYDQVCFQPNSGAQGEYAGLATIRAYLNQKGEGHRTVCLIPKSAHGTNPASAHMAGMKIQPVEVDKYGNIDAVHLKAMVDKHKENLAAIMITYPSTNGVFEENISDVCDLIHQHGGQVYLDGANMNAQVGICRPGDFGSDVSHLNLHKTFCIPHGGGGPGMGPIGVKKHLAPFLPNHPVISLKRNEDACPVGTVSAAPWGSSSILPISWAYIKMMGGKGLKQATETAILNANYMAKRLETHYRILFRGARGYVGHEFILDTRPFKKSANIEAVDVAKRLQDYGFHAPTMSWPVAGTLMVEPTESEDKAELDRFCDAMISIRQEIADIEEGRIDPRVNPLKMSPHSLTCVTSSHWDRPYSREVAAFPLPFVKPENKFWPTIARIDDIYGDQHLVCTCPPMEVYESPFSEQKRASS

SEQ ID NO: 13 - protein, 413 aa, Artificial Sequence
  Description of Artificial Sequence Synthetic consensus polypeptide
MRGGSLXXXXXXXXXXXXXXXXXXXXXXXRXXXXXXXXXXXXTXLXXXHXXXGGXMVXFAGWXXPXQYXXXXXXSXXXXRXXXSXFDVXHMXXXXXXGXDXXXXXEXXVVXDXAXLXXXXGXLXXXTNEXGXXXDDXXXXXXXXXXXYXVXNAGCXXKDXXXXXXXXXXXXXXGXDVXXXXXXXRXXLXXQGPXXXXVLQXXXXXDXXKLXFXXXXXXXXXGXXXCXXTRXGYTGEDGXEISVPXXXAVXLAXXXLXXXXGKVXXXGLXARDSLRLEAGLCLYGNDXXXXXXPVEXXLXWXXGKRRRXXXXFXGAXXIXXQXKXXXXXXRVGXXXXGXPXRXXXXXXXXXGXXXGXXTSGXXSPXLXXNXAMGYVXXXXXXXGTXXXXXVRXKXXXXXXXKMPFVXXXYYXXX

SEQ ID NO: 14 - protein, 403 aa, Homo sapiens
  MOD_RES (4)..(4): Any amino acid
MQRXVSVVARLGFRLQAXPPAXCRPLSXAQXVLRRTPLYDFHLAHGGKMVAFAGWSLPVQYRDSHXDSHLHTRQHCSLFDVSHMLQTKIXGXDRVKLMESLVVGDIAELRPNQGTLSLFTNEAGGILDDLIVTNTSEGHLYVVSNAGCXEKDLALMQDKVRELQNXGRDVGLEVLDNALLALQGPTAAQVLQAGVADDLXKLPFMTSAVMEVFGVSGCRVTRCGYTGEDGVEISVPXAXAVHLATAXLKNPEVKLAGLAARDSLRLEAGLCLYGNDIDEHTTPVEGSLSWTLGKRRRAAMDFPGAKVIVPQLKGXVQRRRVGLMCEGAPXRAHSPILNXEGTXIGTVTSGCPSPSLKKNVAMGYVPCEYSRPGTMLLVEVRRKQQMAVVSKMPFVPTNYYTLK

SEQ ID NO: 15 - protein, 403 aa, Macaca mulatta
  MOD_RES (4)..(4): Any amino acid
MQRXVSVVAXLGFRLQALPPAXCRPLSXAQDVLRRTPLYDFHLAHGGKMVAFAGWSLPVQYRDSHXDSHLHTRQHCSLFDVSHMLQTKIFGXDRVKLMESLVVGDIAELRPNQGTLSLFTNEAGGILDDLIVTNTSEGHLYVVSNAGCXEKDLALMQDKVRELQNXGRDVGLEVLDNALLALQGPTAAQVLQAXVADDLXKLPFMTSAVMEVFGVSGCRVTRCGYTGEDGVEISVPAAXAVHLATALLKNPEVKLAGLAARDSLRLEAGLCLYGNDIDEHTTPVEGSLSWTLGKRRRAAMDFPGAKVIVPQLKGKVQRRRVGLMCEGAPXRAHSPILNXEGTXIGTVTSGCPSPSLKKNVAMGYVPCEYSRPGTMLLVEVRRKQQMAVVSKMPFVPTNYYTLK

SEQ ID NO: 16 - protein, 397 aa, Bos taurus
  MOD_RES (1)..(1): Any amino acid
XVXRLXXRLQALXXAXCRXLSXAQDVLXRTPLYDFHLAHGGKMVAFAGWSLPVQYRDSHXXSHLHTRQHCSLFDVSHMLQTKIFGXDRVKLMESLVVGDIAELXPNQGTLSLFTNEAGGILDDLIVTXXSEGHLYVVSNAGCREKDLXLMQDKVRELQNXGXDVXLEVXDNALLALQGPTAAQVLQAGVADDLXKLPFMTSAVMEVFGVSGCRVTRCGYTGEDGVEISVPAAXAVHLAXALLKNPEVKLAGLAARDSLRLEAGLCLYGNDIDEHTTPVEGSLSWTLGKRRRAAMDFPGAXVIVPQLKXKXQRRRVGLMCXGAPVRAXSPILXXEGTXIGXVTSGCPSPXLKKNVAMGYVPXEYSRPGTXLLVEVRRKQQXAVVSKMPFVXTNYYXLK

SEQ ID NO: 17 - protein, 393 aa, Gallus sp.
  MOD_RES (1)..(2): Any amino acid
XXRXXXXXXXXXRXLSXAXXXLXXTPLXXXHXAXGGXMVXFAGWSLPVQYXXXHXXSHLHTRXHCSLFDVSHMLQTXXXGXDRVXXXESLVVGDIAELRPXQGTLXLXTNEXGXIXDDLIVTNTXEXHLYVVSNAGCXXKDXAXMXXXXXELXXXGXDVXLEVXXXXAXXXXQGPXXAQVLQAGXXDDLXKLXFMTSXXXXVFGVXGCRVTRCGYTGEDGVEISVPAXXAVXLAXXLLXXPEVXXAGLAARDSLRLEAGLCLYGNDIDEXTTPVEXXLXWTLGKRRRXAMDFPGAXXIXXQXKXKXXRXRVGLXXXGXPXRXXXXILXXEGTXXGTVTSGCPSPSLXKNXAMGYVXXXXSRPGTXLXVEVRXKQXXAXVXKMPFVPTXYYXXK

SEQ ID NO: 18 - protein, 403 aa, Mus musculus
  MOD_RES (2)..(2): Any amino acid
MXRXVSVVAXLGFRLQAXPXXXXRPLSXXQDVLRRTPLYDFHLAHGGKMVAFAGWSLPVQYRDSHXDSHLHTRXHCSLFDVSHMLQTKIFGXDRVKLXESXVVGDIAELRPNQGTLSLFTNEAGGILDDLIVXNTSEGHLYVVSNAGCRXKDLALMQDKVXEXQNXGXDVGLEVXXNALLALQGPTAXQVLQAGVXDDXXKLPFMTSAVMEVFGVSGCRVTRCGYTGEDGVEISVPAXXAVHLATXLLKNPEVKLAGLAARDSLRLEAGLCLYGNDIDEHTTPVEGSLSWTLGKRRRXAMDFPGAKXIVPQLKGXVQRRRVGLXCEGAPVRAHSPILNXEGTXIGTVTSGCPSPSLKKNVAMGYVPXXYSRPGTXLLVEVRRKQQMXVVSKMPFVPTNYYTLK

SEQ ID NO: 19 - protein, 404 aa, Xenopus sp.
  MOD_RES (4)..(16): Any amino acid
MQRXXXXXXXXXXXXXAXXXXXXRXXSXXQXXXXRXTPLYDFHXXHGGKMVXFAGWXLPVQYXDSHXXSHLHTRQHCSXFDVSHMLQTKXXGXDRXXXMESXVVXDIAELXXNQGTLSLFTNEXGGIXDDLIVTXTSXGXLYVVSNAGCXEKDXAXMXXKXXXXXXXGRDVXLEXXDXALLAXQGPXXAXVLQAGXXDDLXKLXFMTSXXXXVFGXXGCRVTRCGYTGEDGVEISVPAXXAVXLAXXLLXNXXVKLAGLAARDSLRLEAGLCLYGNDIDEXTTPVEXSLXWTLGKRRRXAMDFPGAXVIVPQXKGKVXXXRVGLXXXGXPVRXHXPILNXEGXXIGXVTSGCPSPSLXXNVAMGYVXXEYXXXGTXXXXEVRXKXXXXVXXKMPFVPXXYYTLK

SEQ ID NO: 20 - protein, 413 aa, Arabidopsis thaliana
  MOD_RES (7)..(7): Any amino acid
MRGGSLXQXXXSXXXRLXXXXQXXXXXXXRXXXXXXXDXLXXTXLYDFHXAHGGKMVXFAGWSXPXQYXDSXXDSXXXXRXXXSLFDVXHMXXXXXXGXDXVXXXEXLVVXDXAXLXPXXGXLXXFTNEXGGXXDDXXXTXXXXXHXYXVXNAGCRXKDLAXXXXXXXXXXXXGXDVXXXXXDXXXLLALQGPXAAXVLQXXXXXDLXKLXFXXXXXXXXXGXSXCXXTRXGYTGEDGXEISVPXXXAVXLAXAXLXXXEXXVXLXGLXARDSLRLEAGLCLYGNDXXXHXXPVEXXLXWXXGKRRRAXXXFXGAXVIXXQLKXXXXXRRVGXXXXGXPXRXHSXXXXXXGXXIGXXTSGXXSPXLKKNXAMGYVXXXXXXXGTXXXXXVRXKXXXXXXXKMPFVXTXYYXXX

(19) United States
(12) Patent Application Publication
(10) Pub. No.: US 2013/0288993 A1
(43) Pub. Date: Oct. 31, 2013

(54) METHOD FOR DIAGNOSIS AND METHOD OF TREATMENT OF AUTISM SPECTRUM DISORDERS AND INTELLECTUAL DISABILITY

(71) Applicants: Christopher A. Walsh, Chestnut Hill, MA (US); Ganeshwaran H. Mochida, Brookline, MA (US); Tim W. Yu, Boston, MA (US); Maria H. Chahrour, Boston, MA (US)

(72) Inventors: Christopher A. Walsh, Chestnut Hill, MA (US); Ganeshwaran H. Mochida, Brookline, MA (US); Tim W. Yu, Boston, MA (US); Maria H. Chahrour, Boston, MA (US)

(73) Assignee: CHILDREN'S MEDICAL CENTER CORPORATION, Boston, MA (US)

(21) Appl. No.: 13/991,451

(22) PCT Filed: Dec. 6, 2011
(86) PCT No.: PCT/US11/63489

§ 371 Date: Jul. 16, 2013

Related U.S. Application Data
(60) Provisional application No. 61/419,908, filed on Dec. 6, 2010.

(51) Int. Cl. C12Q 1/68

(52) U.S. Cl. CPC C12Q 1/6883
USPC 514/43; 435/6.11; 536/24.31; 514/557; 514/575

(57) ABSTRACT

We provide a set of novel mutations in HIST3H3, AMT, GLDC and PEX7 genes which we have discovered as causative of some autism spectrum disorders and/or intellectual disability after analysis of families with more than one affected child and with consanguineous parents. Based on some of these mutations, we also provide novel treatment options for autism spectrum disorders and/or intellectual disability wherein the novel mutations have been diagnosed. The invention is based on the discovery that certain specific mutations, particularly when present in a homozygous, compound heterozygous, or trans heterozygous combinations, result in a phenotype of an autism spectrum disorder and/or intellectual disability. Some mutations also cause the disorder or disease as heterozygous mutation.

### CROSS-REFERENCE TO RELATED APPLICATIONS

This application claims benefit under 35 U.S.C. 119(e) of a U.S. provisional application No. 61/419,908 filed on Dec. 6, 2010, the contents of which are herein incorporated by reference in their entirety.

### GOVERNMENT SUPPORT

This application was supported by the Government with a grant number T32 NS007484-08 and contract numbers HHSN268200782096C and NIH N01-HG-65403 awarded by the National Institutes of Health. The Government has certain rights to the invention.

### SEQUENCE LISTING

The instant application contains a Sequence Listing which has been submitted in ASCII format via EFS-Web and is hereby incorporated by reference in its entirety. Said ASCII copy, created on Dec. 6, 2011, is named 69201PCT.txt and is 102,210 bytes in size.

### BACKGROUND OF THE INVENTION

Autism, autism spectrum disorders are a clinically heterogeneous condition characterized by defects in socialization and language. Despite strong evidence for high heritability in autism, specific genetic causes are identifiable in <15% of cases, likely reflecting underlying genetic heterogeneity. The majority of known autism genes have been discovered on the basis of their disruption by spontaneous mutation, commonly as chromosome rearrangements, while the contribution of recessive mutations remains to be established (Mitchell. The genetics of neurodevelopmental disease, Current Opinion in Neurobiology, 21(1): 197-203, 2011).

Autism spectrum disorders and intellectual disability are genetically and phenotypically variable disorders for which additional diagnostic tests would be useful as identification of different mutations may not only assist in diagnostic screenings, prenatal and pre-implantation diagnostic but also indicate different treatment options for autism spectrum disorders.

### SUMMARY OF THE INVENTION

We provide a set of novel mutations which we have discovered as causative of some autism spectrum disorders and/or intellectual disability with the assistance of families with consanguineous parents. Based on these mutations, we also provide novel treatment options for autism spectrum disorders and/or intellectual disability wherein the novel mutations have been diagnosed. The invention is based on the discovery that certain specific mutations, particularly when present in a homozygous, compound heterozygous, or trans heterozygous combinations, result in a phenotype of an autism spectrum disorder and/or intellectual disability.

Specifically, in one embodiment, the invention provides an in vitro assay comprising a step of analyzing a biological sample from a human individual for at least one mutation in HIST3H3 gene, AMT gene, GLDC gene or PEX7 gene, wherein a homozygous nucleic acid mutation resulting in an amino acid mutation selected from R54H, R129C, or R130C in a HIST3T3 protein or E211K in a AMT protein; a compound heterozygous mutation resulting in any one of the amino acid mutation combinations of L90F/V705M, L90F/G18C, or A569T/A97V in a GLDC protein; or a heterozygous mutation resulting in an amino acid mutation W75C in a PEX7 protein or a heterozygous amino acid mutation I308F in the AMT protein indicates that the autism spectrum disorder and/or intellectual disability in the individual is caused by the identified mutation or mutations. The biological sample may comprise proteins or nucleic acids, such as DNA or RNA.

In some aspects of this and all the other embodiments and aspects of this invention, the in vitro assay further comprises a step of determining whether or not a histone modulating agent is useful as an optional treatment for the individual, wherein the presence of the mutation in the HIST3H3 gene that results in a homozygous mutation R54H, R129C, or R130C in the HIST3H3 protein indicates that histone modulating agents are useful as an optional treatment for the individual, and wherein the absence of the mutation in the HIST3H3 gene that results in a homozygous mutation R54H, R129C, or R130C in the HIST3H3 protein indicates that the histone modulating agents are not useful as an optional treatment for the individual.

In some aspects of this and all the other embodiments and aspects of this invention, prior to the step of determining the individual has been assessed by a clinical evaluation and considered as having clinical symptoms of autism spectrum disorder and/or intellectual disability.

In some aspects of this and all the other embodiments and aspects of this invention, the step of analyzing comprises contacting the biological sample with at least one probe which forms a complex with its target nucleic acid or protein and is therefore capable of detecting at least one of the nucleic acid mutations or amino acid mutations.

In some aspects of this and all the other embodiments and aspects of this invention, the probe is a nucleic acid.

In some aspects of this and all the other embodiments and aspects of this invention, the probe is an antibody.

In some aspects of this and all the other embodiments and aspects of this invention, the step of analyzing comprises a step of nucleic acid amplification and/or nucleic acid sequencing.

In some aspects of this and all the other embodiments and aspects of this invention, the assay is an immunoassay, such as ELISA.

In some aspects of this and all the other embodiments and aspects of this invention, the step of analyzing comprises a computer implemented analysis of one or more sequences, e.g., nucleic acid or amino acid sequences, wherein the analysis comprises comparing sequence information from the biological sample to a reference and/or displaying the result of a comparison.

In one embodiment, the invention provides an in vitro assay for prenatal diagnosis of a fetus or pre-implantation diagnosis of an embryo for autism spectrum disorder and/or intellectual disability comprising analyzing a biological sample comprising fetal or pre-implantation embryonic nucleic acids for a mutation in HIST3H3 gene, AMT gene, GLDC gene or PEX7 gene, wherein a homozygous mutation resulting in an amino acid change of any one of R54H, R129C, and R130C in a HIST3T3 protein and E211K in a AMT protein; a compound heterozygous mutation resulting in an amino acid change combination of any one of L90F/V705M, L90F/G18C, and A569T/A97V in a GLDC gene; or an amino acid change of W75C in a PEX7 protein or I308F in the AMT protein is indicative that the fetus or the pre-implantation embryo is affected with autism spectrum disorder and/or intellectual disability.

In some aspects of this and all the other embodiments and aspects of this invention, the step of analyzing comprises nucleic acid sequencing of the HIST3H3 gene, AMT gene, GLDC gene or PEX7 gene or a portion of said genes.

In some aspects of this and all the other embodiments and aspects of this invention, the step of analyzing comprises contacting the fetal nucleic acid with at least one probe capable of hybridizing to one or more of the mutant forms of the HIST3H3 gene, AMT gene, GLDC gene and/or PEX7 gene.

In some aspects of this and all the other embodiments and aspects of this invention, the probe is attached to a solid surface.

In some aspects of this and all the other embodiments and aspects of this invention, the step of analyzing comprises a computer readable medium that allows automatic, computerized, non-human performed comparison of information from the nucleic acid sample with a reference and/or an automatic display of the identified mutations if any.

In some aspects of this and all the other embodiments and aspects of this invention, wherein the in vitro assay further comprises a step of implanting the embryo if the embryo is a homozygous for the wild type allele R54, R129, and R130 in a HIST3T3 protein and E211 in a AMT protein; a L90/V705, L90/G18, and A569/A97 in a GLDC gene; or W75 in a PEX7.

In another embodiment, the invention provides an in vitro assay for determining an optional therapeutic intervention for an individual for the treatment of autism spectrum disorder and/or intellectual disability comprising the steps of analyzing a biological sample obtained from the individual by contacting the biological sample with at least one probe capable of detecting a nucleic acid mutation resulting in R54H, R129C, or R130C amino acid mutation in HIST3H3 gene, wherein if the mutation is detected and is homozygous, the individual is determined as a candidate for an optional therapeutic intervention with a histone modulating agent.

In yet another embodiment, the invention provides a method of treating autism spectrum disorder and/or intellectual disability comprising the steps of (a) determining if the individual is homozygous for a mutation in the HIST3H3 gene resulting in a homozygous amino acid change R54H, R129C, or R130C in the HIST3H3 protein; and (b) administering a histone modulating agent to the individual if the individual is homozygous for a mutation in the HIST3H3 gene resulting in an amino acid change R54H, R129C, or R130C in the HIST3H3 protein.

The invention also provides a nucleic acid array comprising at least one probe to detect at least one mutation or a pair of mutations selected from a HIST3H3 gene resulting in an amino acid change R54H, R129C, or R130C in a HIST3H3 protein, a mutation in an AMT gene resulting in an amino acid change E211K in an AMT protein; mutations in an GLDC gene resulting in a pair of amino acid changed L90F/V705M, L90F/G18C, or A569T/A97V in an GLDC protein; and a mutation in a PEX7 gene resulting in an amino acid change W75C in a PEX7 protein.

The invention further provides a kit for the diagnosis of autism spectrum disorder and/or intellectual disability comprising at least one probe to detect at least one mutation or a pair of mutations selected from a HIST3H3 gene resulting in an amino acid change R54H, R129C, or R130C in a HIST3H3 protein, a mutation in an AMT gene resulting in an amino acid change E211K in an AMT protein; a mutation in an GLDC gene resulting in a pair of amino acid changed L90F/V705M, L90F/G18C, or A569T/A97V in an GLDC protein; and a mutation in a PEX7 gene resulting in an amino acid change W75C in a PEX7 protein for the diagnosis of autism spectrum disorder and/or intellectual disability.

In some aspects of this and all the other embodiments and aspects of this invention, the probe is attached to a solid surface.

In some aspects of this and all the other embodiments and aspects of this invention, the probe is an antibody.

In some aspects of this and all the other embodiments and aspects of this invention, the probe is a nucleic acid.

The amino acid numbering in the HIST3H3 protein used in the claims relates to the amino acid sequence set forth in SEQ ID NO: 2, which is based on the sequence identified as NM_{—}003493.2 in the Examples.

The invention also provides a method of detecting presence of at least one mutant protein in a biological sample comprising contacting a test sample of tissue cells from a human having clinical symptoms of autism spectrum disorder and/or intellectual disability with an antibody that specifically binds a protein comprising a mutant amino acid sequence set forth in the present application, i.e. binds to the mutant protein with at least 10-50% or more effectively compared to a protein that has the wild-type sequence, and detecting the formation of a complex between said antibody and said protein in the test sample,

### BRIEF DESCRIPTION OF DRAWINGS

FIGS. 1A-1C show a pedigree (FIG. 1A) of the MC-9200 family with children affected with autism spectrum disorder marked with filled circles (females) and filled squares (males). The open circles represent phenotypically non-affected females and open squares represent phenotypically non-affected males. FIG. 1B shows chromosome land the arrow points to the region wherein the linkage was identified; mutations identified in the HIST3H3 gene are marked below the chromosome. FIG. 1C shows mRNA expression analysis of HIST3H3 gene in fetal brain and adult brain showing that the gene is much more expressed in the adult brain. The values were normalized against housekeeping gene GAPDH expression in the same tissues.

FIGS. 2A-2C demonstrate the mutation analysis and show the raw data from the analysis of the HIST3H3 gene in three different families, namely, MC-9200 (FIG. 2A); AU-8600 (FIG. 2B); and AU-5900 (FIG. 2C). The numbering of these mutations in this figure is based on amino acid numbering wherein the first Methionine is denoted with “0”. Thus, in the SEQ ID NO: 1, wherein the numbering begins denoting the first Methionine with “1”, the mutations are located at R130C (FIG. 2A); R129C (FIG. 2B); and R54H (FIG. 2C).

FIG. 3 depicts an amino acid alignment of the AMT gene's conserved regions in human (SEQ ID NO: 14), macaque (SEQ ID NO: 15), cow (SEQ ID NO: 16), chick (SEQ ID NO: 17), mouse (SEQ ID NO: 18), xenopus (SEQ ID NO: 19) and Arabidopsis (SEQ ID NO: 20). SEQ ID NO: 13 indicates the consensus sequence shown on the top of the alignment.

FIG. 4 shows Table 1 detailing results from a population screening and indicating the number (#) of cases in AGRE or AGRE+AC families with novel variants not found in controls. Also listed in the number of novel variants not found in cases.

### DETAILED DESCRIPTION OF THE INVENTION

All the references cited herein and throughout the specification are herein incorporated by reference in their entirety.

We provide novel genes and mutations for diagnosis of autism spectrum disorders and/or intellectual disability.

For example, HIST3H3 is a human gene that encodes for a key component of the chromatin assembly complex, which controls which genes are active at any given time in any given tissue. We have identified three specific mutations in this gene from three families affected by intellectual disability and/or autism spectrum disorders. We have shown, using both statistical methods and biochemical analysis that these mutations cause the autism spectrum disorder and/or intellectual disability in these families.

We believe that this is the first report of human intellectual disability and/or autism, associated with mutations of a histone gene. The mutations we identified in the HIST3H3 gene disrupt key arginine residues that are crucial for the function of this gene, namely gene mutations resulting in amino acid mutations R54, R129 and R130.

Accordingly, we provide a novel addition to genetic screens of autism spectrum diseases by inclusion HIST3H3 probes or antibodies, that identify mutations in locations R54, R129 and R130 of the HIST3H3 protein, for example nucleic acid mutations resulting in amino acid changes R54H, R129C or R130C. From our results, one can also soundly predict that other mutations that similarly disrupt the function of the HIST3H3 gene, such as mutations substituting the critical Arginine residues, in the same positions with an amino acid other than Cysteine, can be screened for in patients with autism spectrum disorders and/or intellectual disability as causative mutations, particularly if they are identified in homozygous form. Accordingly, we also provide mutations that substitute the R54, R129 and R130 with a non-basic amino acid, selected from aspartic acid—asp—D; cysteine—cys—C; glutamine—gln—Q; glutamic acid—glu—E; glycine—gly—G; isoleucine—ile—I; leucine—leu—L; methionine—met—M; phenylalanine—phe—F; proline—pro—P; serine—ser—S; threonine—thr—T; tryptophan—trp—W; tyrosine—tyr—Y and valine—val—V; and optionally also alanine—ala—A.

The assays and methods provided allow accelerated diagnosis of individuals with intellectual disability and/or autism spectrum disorder, and enable early intervention and treatment, for example, in children. Also, in parents having one or more children with autism spectrum disorder and/or intellectual disability, the methods allow prenatal or pre-implantation diagnostics. For example, if the parents opt for in vitro fertilization, embryos that do not carry a homozygous mutation in the HIST3H3 gene can be selected for implantation over embryos that carry homozygous mutations.

Furthermore the particular mutations we have discovered that affect HIST3H3 in patients with autism spectrum disorders and/or intellectual disability provide novel therapeutic interventions using chromatin modifying agents, such as histone deacetylases and acetyltransferases. Accordingly, we also provide novel therapeutic interventions for individuals who carry these mutations as the individuals can be administered modifiers of histone deacetylases and/or acetyltransferases to ameliorate the symptoms of autism spectrum disorder and/or intellectual disability. These modifiers can be administered in conjunction with other therapies known to be of assistance in treatment of autism spectrum disorders.

Various histone modifying drugs have been developed and are currently either in clinical trials or in use. The following is a list of such therapeutic agents that can be used for treatment of autism spectrum disorders and/or intellectual disability: Vorinostat (SAHA) (FDA approved), Belinostat, LAQ824, Panobinostat, Pyroxamide, Givinostat, PCI-24781, Romidepsin, AN 9, Sodium Phenylbutyrate, Valproic acid, BACECA®, SAVICOL™, Entinostat, Tacedinaline, MGCD 0103, DACOGEN™, VIDAZA® (FDA approved), ZOLINZA® (FDA approved), Anacardic acid, Curcumin, Isothiazolones, Garcinol, MB-3, H3-CoA-20, AMI-1, AMI-5, Stilbamidine, and DZNep. Dosages can be determined empirically based on the known dosages that are currently used, the age, weight and other parameters of the patient as well as observing whether the symptoms are ameliorated or not, and whether the side effects may be less or more with a particular dosage. The dosage adjustments are routine and can be performed with the existing quidange regarding the use of these drugs.

We have also identified several additional mutations in PEX7, AMT, GLDH genes that cause autism spectrum disorder and/or intellectual disability when present either in heterozygous form, in a compound homozygous form or in homozygous form. These mutations can also affect the phenotype in trans heterozygous combinations with other mutations. Accordingly, we also provide assays, kits and methods for detecting mutations in PEX7, AMT, GLDH genes.

We used high-throughput DNA sequencing to study patients whose parents share ancestry to identify recessive mutation associated with autism. We identify multiple examples by which recessive mutations cause familial autism: mutations in HIST3H3, a brain-expressed gene that regulates chromatin structure, and mild, “hypomorphic” mutations in AMT and PEX7, two genes traditionally associated with neurometabolic disease syndromes. We also found evidence in non-consanguineous autism cases for an unappreciated burden of mild metabolic disease via copy number deletion, mild recessive mutation or transheterozygous noncomplementation. Extending these results, whole-exome sequencing in additional autism cases from nonconsanguineous populations reveals a rich burden of potentially pathogenic recessive mutation, from which we identified a single autism candidate gene that segregates with the disease in several families.

Our data show that individually rare recessive mutations are an important contributor to the burden of autism, and provide novel approaches to identifying additional mutations in the genes function disrupting mutations of which we have now identified as causative of some forms of autism spectrum disorders. In view of our data, we also provide several new therapeutic targets, including glycine metabolism and histone biology.

In sequencing controls and AGRE samples available in our laboratory, we identified mutations in the HIST3H3 resulting in amino acid chases as set forth in Table 1.

TABLE 1

| Six Caucasian control | |
|---|---|
| plates (532 samples | |
| were successfully | Identified mutations |
| sequenced) | in HIST3H3 gene |
| | Heterozygous (Het) R53H (i.e. R54H when |
| | reading from the SEQ ID NO: 1) in one |
| | sample |
| | Het A1V (i.e. A2V when reading from the |
| | SEQ ID NO: 1) in one sample |
| | Het R2X (i.e. R3X when reading from the |
| | SEQ ID NO: 1) in one sample |
| 521 AGRE samples | Het A7T (i.e. A8T when reading from the |
| | SEQ ID NO: 1) in four samples |
| | Het K36Q (i.e. K37Q when reading from the |
| | SEQ ID NO: 1) in one sample |
| | Het D77E (i.e. D78E when reading from the |
| | SEQ ID NO: 1) in one sample |

Table 2 in shows whole exome sequencing that identified novel homozygous variants in 18 AGRE patients.

TABLE 2

| | | | ROH size |
|---|---|---|---|
| | | | (cM = |
| Gene | Amino acid change | Gene description | centi Morgan) |
| BANP | P > S | Btg3 associated | 0.40 |
| | | nuclear protein | |
| | | isoform b | |
| C10orf125 | Y > STOP | Chromosome 10 | 0.51 |
| | | open reading frame | |
| | | 125 | |
| KIF26A | R > C | Kinesin family | 3.44 |
| | | member 26A | |
| PCDHB10 | E > Q | Protocadherin beta | 1.90 |
| | | 10 precursor | |
| PTPRH | Q > STOP | Protein tyrosine | 8.84 |
| | | phosphatase, receptor | |
| | | type, H | |
| SF3A1 | G > STOP | Splicing factor 3a, | 1.06 |
| | | subunit 1, isoform 1 | |
| SLC25A1 | A > E | Solute carrier family | 0.49 |
| | | 25 | |
| WDR85 | R > Q | WD repeat- | 3.26 |
| | | containing protein 85 | |

### HIST3H3 Gene and Mutations

Histones are basic nuclear proteins that are responsible for the nucleosome structure of the chromosomal fiber in eukaryotes. Nucleosomes consist of approximately 146 bp of DNA wrapped around a histone octamer composed of pairs of each of the four core histones (H2A, H2B, H3, and H4). The chromatin fiber is further compacted through the interaction of a linker histone, H1, with the DNA between the nucleosomes to form higher order chromatin structures. This gene is intronless and encodes a member of the histone H3 family. Transcripts from this gene lack polyA tails; instead, they contain a palindromic termination element. HIST3H3 gene is located in chromosome 1, separately from the other H3 genes that are in the histone gene cluster on chromosome 6p22-p21.3.

The present application identifies HIST3H3 DNA as SEQ ID NO: 1 and HIST3H3 protein as SEQ ID NO: 2.

We identified three homozygous Arginine (R) to Cytosine (C) mutations in locations where the Arginine residues are known to play a critical role. The homozygous mutations affected the Arginines in locations R54 in SEQ ID NO: 2, R129C in SEQ ID NO: 2, and R130C in SEQ ID NO:2. We originally numbered these mutations according to a sequence where the initial Methionine residue was not counted and the sequence of the HIST3H3 began from the first Alanine residue in the HIST3H3 protein. Accordingly, our initial results presented in the provisional application No. 61/419,908 showed these mutations in locations R53H (SEQ ID NO:6), R128C (SEQ ID NO: 7) and R129C (SEQ ID NO: 8).

Similarly, mutations in the same locations R54, R129 and R130 substituting the R with a non-basic amino acid, selected from aspartic acid—asp—D; cysteine—cys—C; glutamine—gln—Q; glutamic acid—glu—E; glycine—gly—G; isoleucine—ile—I; leucine—leu—L; methionine—met—M; phenylalanine—phe—F; proline—pro—P; serine—ser—S; threonine—thr—T; tryptophan—trp—W; tyrosine—tyr—Y and valine—val—V; and optionally also alanine—ala—A, are contemplated in the assays, methods, arrays and kits of the invention.

### AMT Aminomethyltransferase, Mitochondrial Isoform 1 Precursor Gene and Mutations

AMT gene encodes one of four critical components of the glycine cleavage system. Mutations in the AMT gene have been previously associated with glycine encephalopathy. Multiple transcript variants encoding different isoforms have been found for this gene. AMT sequence variant we identified the mutation is indicated as follows: DNA is disclosed herein as SEQ ID NO: 9 and AMT protein is disclosed as SEQ ID NO: 10. The sequences variants listed here refer to these specific reference SEQ ID NOs. We identified nucleic acids changes resulting in homozygous amino acid substitution E211K in the AMT protein, and a homozygous or heterozygous I308Fsubstitution in the AMT protein.

Similarly, other mutations resulting in different amino acid substitutions in these specific locations of E211 and I308 are contemplated.

Particularly, because glutamic acid (E) is acidic, mutations resulting in non-acidic substitutions of E211 are contemplated as disease causing mutations. These include, aliphatic amino acids alanine, glycine, isoleucine, leucine, proline, valine; aromatic amino acids including phenylalanine tryptophan, tyrosine; basic amino acids including arginine, histidine, lysine; hydroxylic amino acids including serine, threonine; sulphur-containing amino acids including cysteine, and methionine; and amidic (containing amide group)-asparagine, and glutamine.

For substituting I308, isoleucine being aliphatic, non-aliphatic amino acid mutations are contemplated as disease causing mutations that can be included into the assays, methods, kits and arrays of the invention. These include substitutions of I308 with aromatic amino acids including phenylalanine tryptophan, tyrosine; basic amino acids including arginine, histidine, lysine; hydroxylic amino acids including serine, threonine; sulphur-containing amino acids including cysteine, and methionine; amidic (containing amide group)-asparagine, and glutamine; and acidic amino acids including aspartic acid and glutamic acid.

### GLDC Glycine Dehydrogenase (Decarboxylating) Gene and Mutations

Degradation of glycine is brought about by the glycine cleavage system, which is composed of four mitochondrial protein components: P protein (a pyridoxal phosphate-dependent glycine decarboxylase), H protein (a lipoic acid-containing protein), T protein (a tetrahydrofolate-requiring enzyme), and L protein (a lipoamide dehydrogenase). The protein encoded by GLDC glycine dehydrogenase gene is the P protein, which binds to glycine and enables the methylamine group from glycine to be transferred to the T protein. Specific defects in this protein have been previously associated with non-ketotic hyperglycinemia (NKH). The DNA sequence for GLDC is disclosed herein as SEQ ID NO: 11 and the GLDC protein is disclosed herein as SEQ ID NO: 12.

We identified three different causative compound heterozygous mutations in individuals with autism spectrum disorder and/or intellectual disability, wherein the two different mutations, one in each allele of the gene, result in two protein variants being expressed in the patient, namely, in one instance, one carrying an amino acid substitution L90F and another an amino acid substitution V705M; in another instance, one carrying an amino acid substitution L90F and another carrying an amino acid substitution G18C; and in yet another instance, one carrying an amino acid substitution A569T and another carrying an amino acid substitution A97V in the GLDC protein.

Similarly as described in the case of HIST3H3 and AMT mutations substitutions of L90 or V705 in the GLDC to non-aliphatic amino acids is contemplated.

### PEX7 Gene and Mutations

The PEX7 gene encodes for a protein called peroxisomal biogenesis factor 7, which is part of a group known as the peroxisomal assembly (PEX) proteins. Within cells, PEX proteins are responsible for importing certain enzymes into structures called peroxisomes. The enzymes in these sac-like compartments break down many different substances, including fatty acids and certain toxic compounds. They are also important for the production (synthesis) of fats (lipids) used in digestion and in the nervous system.

Peroxisomal biogenesis factor 7 transports several enzymes that are essential for the normal assembly and function of peroxisomes. The most important of these enzymes is alkylglycerone phosphate synthase (produced from the AGPS gene). This enzyme is required for the synthesis of specialized lipid molecules called plasmalogens, which are present in cell membranes throughout the body. Peroxisomal biogenesis factor 7 also transports the enzyme phytanoyl-CoA hydroxylase (produced from the PHYH gene). This enzyme helps process a type of fatty acid called phytanic acid, which is obtained from the diet. Phytanic acid is broken down through a multistep process into smaller molecules that the body can use for energy.

Mutations in the PEX7 gene cause a small percentage of all cases of Refsum disease. The three mutations known to be responsible for this condition reduce the activity of peroxisomal biogenesis factor 7, which disrupts the import of several critical enzymes (including phytanoyl-CoA hydroxylase) into peroxisomes. Without enough of these enzymes, peroxisomes cannot break down fatty acids and other substances effectively.

In people with Refsum disease, a shortage of phytanoyl-CoA hydroxylase prevents peroxisomes from breaking down phytanic acid. Instead, this substance gradually builds up in the body's tissues. Over time, the accumulation of phytanic acid becomes toxic to cells. It is unclear, however, how an excess of this substance affects vision and smell and causes the other specific features of Refsum disease.

More than three dozen mutations in the PEX7 gene have been found to cause rhizomelic chondrodysplasia punctata type 1 (RCDP1). These mutations tend to be more severe than the mutations that cause Refsum disease. The genetic changes associated with RCDP1 often lead to a completely nonfunctional version of peroxisomal biogenesis factor 7 or prevent cells from making any of this protein. The most common mutation responsible for RCDP1 replaces the amino acid leucine at protein position 292 with a premature stop signal in the instructions for making peroxisomal biogenesis factor 7 (written as Leu292Ter or L292X). This mutation leads to a nonfunctional version of the protein.

PEX7 DNA is disclosed herein as SEQ ID NO: 3 and PEX7 protein is disclosed herein as SEQ ID NO: 4.

We identified a heterozygous causative mutation in the PEX gene substituting the amino acid W75 with a C (W75C, SEQ ID NO: 5). As described in connection with the other mutations, with the same logic, mutations substituting amino acid W75 with at least a non-aromatic amino acid selected from aliphatic—alanine, glycine, isoleucine, leucine, proline, valine; basic—arginine, histidine, lysine; hydroxylic—serine, threonine; sulphur-containing-cysteine, methionine; and amidic (containing amide group)-asparagine, and glutamine are contemplated.

### Biological Samples Useful in the Methods and Assays of the Invention

A “biological sample” as used herein refers to a sample which comprises nucleic acids, such as DNA or total RNA or mRNA or proteins from a human individual subject, fetus or pre-implantation embryo. Typically, if the biological sample is a sample from a fetus or from a pre-implantation embryo, the sample comprises only a few or a single cell. However, in some embodiments, the term also refers to non-cellular biological material, such as plasma, such as in non-invasive prenatal diagnostic methods, wherein the sample is typically maternal blood or plasma. Non-cellular biological samples, such as fractions of blood, saliva, or urine that can be used analyze the presence of absence of the mutations of the present invention. The sample is typically fresh, but can be a sample that has been stored from hours or days, or frozen as well. The frozen sample can be thawed before employing methods, assays and systems of the invention. After thawing, a frozen sample can be centrifuged before being subjected to methods, assays and systems of the invention.

In some embodiments, the test sample or the biological sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. In addition, or alternatively, chemical and/or biological reagents can be employed to release nucleic acid or protein from the sample.

The skilled artisan is well aware of methods and processes appropriate for pre-processing of test or biological samples, e.g., blood, required for determination of nucleic acids, such as DNA or RNA, or proteins comprising the mutations as disclosed herein.

In some embodiments, the test sample or biological sample is a blood sample, e.g., whole blood, plasma, and serum. In some embodiments, the test sample or biological sample is a whole blood sample. In some embodiments, the test sample or biological sample is a serum sample. In some embodiments, the test sample or biological sample is a plasma sample. In some embodiments, the blood sample can be allowed to dry at room temperature from about 1 hour to overnight, or in the refrigerator (low humidity) for up to several months before subjected to analysis, e.g., SNP analysis. See, for example, Ulvik A. and Ueland P. M. (2001) Clinical Chemistry 47: 2050, for methods of SNP genotyping in unprocessed whole blood and serum by real-time PCR.

For example, nucleic acids or proteins can be present in a blood sample. To collect a blood sample, by way of example only, the patient's blood can be drawn by trained medical personnel directly into anti-coagulants such as citrate, EDTA PGE, and theophylline. The whole blood can be separated into the plasma portion, the cells, and platelets portion by refrigerated centrifugation at 3500 g for 2 minutes. After centrifugation, the supernatant is the plasma and the pellet is RBC. Since platelets have a tendency to adhere to glass, it is preferred that the collection tube be siliconized. Another method of isolating red blood cells (RBCs) is described in Best, C A et al., 2003, J. Lipid Research, 44:612-620.

Alternatively, serum can be collected from the whole blood. By way of example, about 15 mL of whole blood can be drawn for about 6 mL of serum. The blood can be collected in a hard plastic or glass tube; blood will not clot in soft plastic. The whole blood is allowed to stand at room temperature for 30 minutes to 2 hours until a clot has formed. Then, clot can be carefully separated from the sides of the container using a glass rod or wooden applicator stick and the rest of the sample can be left overnight at 4° C. After which, the sample can be centrifuged, and the serum can be transferred into a clean tube. The serum can be clarified by centrifugation at 1000 g for 10 minutes at 4° C. The serum can be stored at −80° C. before analysis. In such embodiments, carotenoids may not be stable for long periods of time. Detailed described of obtaining serum using collection tubes can be found in U.S. Pat. No. 3,837,376 and is incorporated by reference. Blood collection tubes can also be purchased from BD Diagnostic Systems, Greiner Bio-One, and Kendall Company.

The whole blood can be first separated into platelet-rich plasma and cells (white and red blood cells). Platelet rich plasma (PRP) can be isolated from the blood centrifugation of citrated whole blood at 200 g for 20 minutes. The platelet rich plasma is then transferred to a fresh polyethylene tube. This PRP is then centrifuged at 800 g to pellet the platelets and the supernatant (platelet poor plasma [PPP]) can be saved for analysis, e.g., by ELISA, at a later stage. Platelets can be then gently re-suspended in a buffer such as Tyrodes buffer containing 1 U/ml PGE2 and pelleted by centrifugation again. The wash can be repeated twice in this manner before removing the membrane fraction of platelets by centrifugation with Triton X, and lysing the pellet of platelet for platelet-derived PF4 analyses. Platelets can be lysed using 50 mM Tris HCL, 100-120 mM NaCl, 5 mM EDTA, 1% Igepal and Protease Inhibitor Tablet (complete TM mixture, Boehringer Manheim, Indianopolis, Ind.).

In one embodiment, platelets are separated from whole blood and the mutations are detected in the platelet sample can be determined therefrom. When whole blood is centrifuged as described herein to separate the blood cells from the plasma, a pellet is formed at the end of the centrifugation, with the plasma above it. Centrifugation separates out the blood components (RBC, WBC, and platelets) by their various densities. The RBCs are denser and will be the first to move to the bottom of the collection/centrifugation tube, followed by the smaller white blood cells, and finally the platelets. The plasma fraction is the least dense and is found on top of the pellet. The “buffy coat” which contains the majority of platelets will be sandwiched between the plasma and above the RBCs. Centrifugation of whole blood (with anti-coagulant, PGE and theophylline) can produce an isolated a platelet rich “buffy coat” that lies just above the buoy. The buffy coat contains the concentrated platelets and white blood cells.

In another embodiment, platelets can be separated from blood according to methods described in U.S. Pat. No. 4,656,035 using lectin to agglutinate the platelets in whole blood. Alternatively, the methods and apparatus described in U.S. Pat. No. 7,223,346 can be used involving a platelet collection device comprising a centrifugal spin-separator container with a cavity having a longitudinal inner surface in order to collect the “buffy coat” enriched with platelets after centrifugation. As another alternative, the methods and apparatus as described in WO/2001/066172 can be used. Each of these references is incorporated by reference herein in their entirety.

In another embodiment, platelets can be isolated by the two methods described in A. L. Copley and R. B. Houlihan, Blood, 1947, 2:170-181, which is incorporated by reference herein in its entirety. Both methods are based on the principle that the platelet layer can be obtained by repeated fractional centrifugation.

If the mutations are detected from an RNA, such as mRNA sample, the methods and assays typically comprise a step of cDNA synthesis. Accordingly, the methods of the invention may include a step of cDNA synthesis prior to amplification of the mRNA.

The assay can be designed to detect one or more of the mutations set forth herein to create a multiplex assay. In the multiplex assay comprising detecting at least two different mutations, one can use the existing probe and primer design software to design primers and probes that are compatible with the assay conditions and that do not interfere with each other, and that allow detection of two or more of the transcripts in one assay.

Moreover, the assays can be combined with other assays, for example for other mutations that are known to cause autism spectrum disorders and/or intellectual disability or other diseases such as diseases typically screened for in a prenatal or preimplantation assays. For example, a microfluidic device can comprise sections for detection of each of the different mutations. Similarly, a microarray or a selection of microbeads comprising probes attached to a solid phase is a convenient way of designing a multi-mutation and/or a multi-disease detection assay.

### Mutation Detection Assays

Any nucleic acid detection method known to one skilled in the art can be used in the assays and methods of the invention. Methods for mutation detection are well known in the art. Detection methods, such as nucleic acid sequencing, solid phase mini-sequencing (Hultman, et al., 1988, Nucl. Acid. Res., 17, 4937-4946; Syvanen et al., 1990, Genomics, 8, 684-692) or allele-specific primer extension, allele-specific nucleic acid amplification, such as PCR, are well known and well described methods that can be used. For review of methods, see, e.g., Louise O'Connor and Barry Glynn, Expert Review of Medical Devices (2010) Volume: 7, Issue: 4, Publisher: Expert Reviews, Pages: 529-539.

The assays and methods may optionally comprise nucleic acid amplification before the mutation detection step. Several different methods of nucleic acid amplification can be used.

The most commonly used method for nucleic acid amplification is the template dependent PCR (Polymerase Chain Reaction). The PCR method enables the exponential amplification a nucleic acid comprising a nucleotide sequence complementary to a template nucleic acid using a small amount the template. In the PCR method, a pair of primers, comprising a complementary nucleotide sequence, are hybridized to both ends of the target nucleotide sequence. The primer pair is designed such that one primer anneals to an extension product provided by another primer. A nucleic acid synthesis reaction proceeds by repeating an annealing to the mutual extension product and a complementary strand synthesis reaction, and an exponential amplification is thus attained.

In general, the PCR procedure describes a method of gene amplification which is comprised of (i) sequence-specific hybridization of primers to specific genes, such as HITS3H3, AMT, GKDC or PEX7 described herein, within a nucleic acid sample, (ii) subsequent amplification involving multiple rounds of annealing, elongation, and denaturation using a DNA polymerase. One can isolate and purify the amplified nucleic acid sample, for example by screening the PCR products for a band of the correct size on a gel and isolating it, or by capturing the PCR product on a bead or an array using, e.g., a biotin/avidin reaction if one of the PCR primers is biotinylated. The primers used are oligonucleotides of sufficient length and appropriate sequence to provide initiation of polymerization, i.e. each primer is specifically designed to be complementary to each strand of the genomic locus to be amplified.

In the PCR method, a single-stranded nucleic acid template is made by some method and a primer is annealed to the template. Since a template dependent DNA polymerase requires a primer as a replication origin, the preparation of the single-stranded template is considered to be essential, in order to anneal the primer to it in the PCR method. The step of converting a double-stranded template nucleic acid to a single-strand is generally called denaturing. The denaturing is usually carried out by heating. Since other reaction components required for the synthesis of nucleic acid, including DNA polymerase, are heat resistant, the denaturing and successive complementary strand synthesis reactions can be carried out by combining all of the reaction components and further heating the reaction mixture.

One can also use methods of amplifying DNA having a complementary sequence to a target sequence using the target sequence as a template, such as the Strand Displacement Amplification (SDA) method (P.N.A.S., 89, pp. 392-396, 1992; Nucleic Acid, Res., 20, pp. 1691-1696, 1992). In the SDA method, when a complementary strand is synthesized using as a synthesis origin a complementary primer to the 3′-side of a certain nucleotide sequence, a unique DNA polymerase enables synthesis of a complementary strand that displaces the double-strand region at the 5′-side. When reciting “5′-side” or “3′-side” hereinafter, the terms mean the direction of a template strand. This method is called Strand Displacement Amplification because the double-strand portion of the 5′-side is displaced with a complementary strand which has been newly synthesized.

In the SDA method, the step of changing temperature, which is essential for the PCR method, can be omitted by inserting a restriction enzyme recognition sequence in a sequence to which a primer anneals. Namely, a nick provided by the restriction enzyme gives a 3′-OH group that becomes the origin of complementary strand synthesis. The strand displacement and complementary strand synthesis are carried out from the origin and the complementary strand synthesized is dissociated as a single-strand and utilized as the template in the subsequent complementary strand synthesis.

Also, one can used a method for amplifying nucleic acid without temperature control, such as, Nucleic Acid Sequence-based Amplification (NASBA), which is also called TMA/Transcription Mediated Amplification method, is known. NASBA is a reaction system in which DNA synthesis is carried out using DNA polymerase, a target RNA as a template, and a probe to which T7 promoter has been added. The synthesized DNA is made double-stranded using a second probe, and transcription is performed using T7 RNA polymerase. The double-stranded DNA obtained is used as a template, thereby amplifying a large quantity of RNA (Nature, 350, pp. 91-92, 1991). Transcription using T7 RNA polymerase in NASBA proceeds isothermally. NASBA uses RNA as a template, and thus be used without the step of cDNA production in the methods of the invention. If cDNA production is used, NASBA reaction can be performed using similar temperature control as used in the PCR.

One can also use Q-beta amplification as described in published European Patent Application (EPA) No. 4544610.

Also, a method called strand displacement amplification (as described in G. T. Walker et al., Clin. Chem. 42: 9-13 (1996) and European Patent Application No. 684315 and be used.

Target mediated amplification, as described by PCT Publication WO 9322461, is yet another method for nucleic acid detection.

Alternatively, nucleic acid synthesis methods using a complementary strand synthesis under a specific condition, using a primer as the origin for the synthesis can be used (WO97/00330). This method recognizes of the fact that the hybridization of nucleic acids having complementary nucleotide sequences occurs in a state of dynamic equilibrium (kinetics). In this method, it is believed that the complementary strand synthesis reaction, using a primer as the origin for the synthesis, may occur at a certain probability, even at a temperature that causes complete denaturing or below. The term “complete denaturing” as used herein means a condition in which most of the double-stranded template nucleic acid becomes single-stranded.

One method to detect the bacterial genes expressed in human blood is loop-mediated isothermal amplification (LAMP)(see, e.g., Notomi et al. Nucl. Acids Res. (2000) 28 (12): e63; and Shaerli et al., Nucl. Acids Res. (2010) 38 (22): e201). In order to achieve complementary strand synthesis using double-stranded nucleic acid as a template without thermal cycling, the complementary strand synthesis reaction using a primer as the origin for the synthesis can be carried out under a constant temperature condition. The known complementary strand synthesis method, based on the dynamic equilibrium between a double-stranded nucleic acid and a primer (WO97/00330), does not require the temperature change. However, it is difficult to attain practically usable synthesis efficiency using this method. Therefore, the method can be combined with the isothermal nucleic acid synthesis reaction in order to efficiently conduct a complementary strand synthesis based on the dynamic equilibrium without deteriorating specificity. As a result, high level amplification efficiency can be achieved.

For example, the amplification can be performed using steps of hybridizing a pair of primers to the nucleic acid to amplify a nucleic acid region where the mutation is located. The primers are typically flanking the region to be amplified. Primers for amplification can be designed using routine methods from the gene sequences provided herein. The amplicons are preferably at least about 50-100 bp long, alternatively about 50-200 bp long and can be up to about 1000 bp long. Longer amplicons or regions can be amplified but the efficiency or the amplification reaction may suffer.

Multiplex amplifications, i.e. amplifications of two or more different nucleic acid regions in the same reaction may also be used to make the analysis.

The mutations in the amplified or non-amplified nucleic acid samples may be detected, e.g., using an allele-specific primer extension reaction and the amplified fragments can be detected using gel electrophoresis, mass spectrometry, such as MALDI TOF, or capture of the labeled amplified products on an array.

The allele-specific primer extension reaction according to the present invention can be performed using any standard base extension method. In general, a nucleic acid primer is designed to anneal to the target nucleic acid next to or close to a site that differs between the different alleles in the locus. In the standard base extension methods, all the alleles present in the biological sample are amplified, when the base extension is performed using a polymerase and a mixture of deoxy- and dideoxcynucleosides corresponding to all relevant alleles. Thus, for example, if the allelic variation is A/C, and the primer is designed to anneal immediately before the variation site, a mixture of ddATP/ddCTP/dTTP/dGTP will allow amplification of both of the alleles in the sample, if both alleles are present.

After the base extension reaction, the extension products including nucleic acids with A and C in their 3′ ends, can be separated based on their different masses. Alternatively, if the ddNTPs are labeled with different labels, such as radioactive or fluorescent labels, the alleles can be differentiated based on the label. In a preferred embodiment, the base extension products are separated using mass spectrometric analysis wherein the peaks representing different masses of the extension products, represent the different alleles.

In one embodiment, the base extension is performed using single allele base extension reaction (SABER). In SABER, one allele of interest per locus is amplified in one reaction by adding only one dideoxynucleotide corresponding to the allele that one wishes to detect in the sample. One or more reactions can be performed to determine the presence of a variety of alleles in the same locus. Alternatively, several loci with one selected allele of interest can be extended in one reaction.

The specificity provided by primer extension reaction, particularly SABER, allows accurate detection of nucleic acids with even a single base pair difference in a sample, wherein the nucleic acid with the single base pair difference is present in very small amounts.

In one embodiment, the primer extension reaction and analysis is performed using PYROSEQUENCING™ (Uppsala, Sweden) which essentially is sequencing by synthesis. A sequencing primer, designed directly next to the nucleic acid differing between the disease-causing mutation and the normal allele or the different SNP alleles is first hybridized to a single stranded, PCR amplified DNA template from the mother, and incubated with the enzymes, DNA polymerase, ATP sulfurylase, luciferase and apyrase, and the substrates, adenosine 5′ phosphosulfate (APS) and luciferin. One of four deoxynucleotide triphosphates (dNTP), for example, corresponding to the nucleotide present in the disease-causing allele, is then added to the reaction. DNA polymerase catalyzes the incorporation of the dNTP into the standard DNA strand. Each incorporation event is accompanied by release of pyrophosphate (PPi) in a quantity equimolar to the amount of incorporated nucleotide. Consequently, ATP sulfurylase converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a PYROGRAM™. Each light signal is proportional to the number of nucleotides incorporated and allows a clear determination of the presence or absence of, for example, the disease causing allele. Thereafter, apyrase, a nucleotide degrading enzyme, continuously degrades unincorporated dNTPs and excess ATP. When degradation is complete, another dNTP is added which corresponds to the dNTP present in for example the selected SNP. Addition of dNTPs is performed one at a time. Deoxyadenosine alfa-thio triphosphate (dATP□S) is used as a substitute for the natural deoxyadenosine triphosphate (dATP) since it is efficiently used by the DNA polymerase, but not recognized by the luciferase. For detailed information about reaction conditions for the PYROSEQUENCING, see, e.g. U.S. Pat. No. 6,210,891, which is herein incorporated by reference in its entirety.

The mutant nucleic acids can be detected using nucleic acid detection in gels, safe imager blue-light transilluminator, SYBR photographic filters, capillary electrophoresis and channel electrophoresis, mass spectrometry such as MALFI TOF, microarrays and blots, and microfluidic devices.

### Mutation Detection Assays Proteins

Based on the mutation, the amino acid changes, one can also analyze the mutant proteins using protein analysis. Routine methods can be used to make antibodies against the mutant proteins. These antibodies can then be screened for their specificity to recognize the mutant protein over the wild type protein. Typically, the binding affinity of the specific antibody is at least 10% more to the mutant protein compared to the wild type protein in assays, such as ELISA. For example, 10-100% increased affinity of the antibody to mutant compared to the wild-type protein is typically useful.

In one embodiment, the invention provides an in vitro assay or method comprising the steps of: (a) contacting in vitro biological sample comprising proteins from a human patient with an isolated and purified first antibody against at least one of the mutant forms of HIST3H3, AMT, PEX7 and/or GLDC or a wild-type equivalent there of or wherein the antibody that recognizes either the mutant or the wild type protein forms a complex with said wild type or mutant protein; and (b) detecting the bound antibody to determine whether the biological sample contains the at one of wild type and/or mutant forms of HIST3H3, AMT, PEX7 and/or GLDC.

Antibodies to mutant and wild-type proteins can be made using routine techniques.

Both polyclonal and monoclonal antibodies can be prepared using the entire proteins as antigens or fragments thereof.

The term “fragment” refers to any subject polypeptide having an amino acid residue sequence shorter than that of a polypeptide whose amino acid residue sequence is described herein.

The fragment preferably comprises at least one epitope. An “epitope” is the collective features of a molecule, such as primary, secondary and tertiary peptide structure, and charge, that together form a site recognized by an immunoglobulin, T cell receptor or HLA molecule. Alternatively, an epitope can be defined as a set of amino acid residues which is involved in recognition by a particular immunoglobulin, or in the context of T cells, those residues necessary for recognition by T cell receptor proteins and/or Major Histocompatibility Complex (MHC) receptors.

Epitopes that comprise the differentiating protein structure, and can be isolated, purified or otherwise prepared/derived by human or non-human means. For example, epitopes can be prepared by isolating the mutant or wild type peptides from a cell culture or prepare using recombinant techniques.

Synthetic epitopes can comprise artificial amino acids “amino acid mimetics,” such as D isomers of natural occurring L amino acids or non-natural amino acids such as cyclohexylalanine. Throughout this disclosure, the terms epitope and peptide are often used interchangeably. In some embodiments, one can use analogs of said epitopes to produce additional antibodies against the mutant and wild-type versions of the proteins described herein.

Protein or polypeptide molecules that comprise one or more peptide epitopes can be used to raise antibodies useful according to the invention. In certain embodiments, there is a limitation on the length of a polypeptide that can be used to make antibodies, for example, not more than 120 amino acids, not more than 110 amino acids, not more than 100 amino acids, not more than 95 amino acids, not more than 90 amino acids, not more than 85 amino acids, not more than 80 amino acids, not more than 75 amino acids, not more than 70 amino acids, not more than 65 amino acids, not more than 60 amino acids, not more than 55 amino acids, not more than 50 amino acids, not more than 45 amino acids, not more than 40 amino acids, not more than 35 amino acids, not more than 30 amino acids, not more than 25 amino acids, 20 amino acids, 15 amino acids, or 14, 13, 12, 11, 10, 9 or 8 amino acids. In some instances, the embodiment that is length-limited occurs when the protein/polypeptide comprising an epitope of the invention comprises a region (i.e., a contiguous series of amino acids) having 100% identity with a native sequence.

An “immunogenic peptide” or “peptide epitope” is a peptide that will bind an HLA molecule and induce a cytotoxic T lymphocyte (CTL) response and/or a helper T lymphocyte (HTL) response. Thus, immunogenic peptides of the invention are capable of binding to an appropriate HLA molecule and thereafter inducing a cytotoxic T lymphocyte (CTL) response, or a helper T lymphocyte (HTL) response, to the peptide.

The term “motif” refers to a pattern of residues in an amino acid sequence of defined length, usually a peptide of from about 8 to about 13 amino acids for a class I HLA motif and from about 16 to about 25 amino acids for a class II HLA motif, which is recognized by a particular HLA molecule. Motifs are typically different for each HLA protein encoded by a given human HLA allele. These motifs often differ in their pattern of the primary and secondary anchor residues.

The term “residue” refers to an amino acid or amino acid mimetic incorporated into a peptide or protein by an amide bond or amide bond mimetic.

“Synthetic peptide” refers to a peptide that is not naturally occurring, but is man-made using such methods as chemical synthesis or recombinant DNA technology.

Antibodies, both polyclonal and monoclonal, can be produced by a skilled artisan either by themselves using well known methods or they can be manufactured by service providers who specialize making antibodies based on known protein sequences. In the present invention, the protein sequences are known and thus production of antibodies against them is a matter of routine.

For example, production of monoclonal antibodies can be performed using the traditional hybridoma method by first immunizing mice with an isolated mutant or wild type protein or fragment thereof of choice wherein the fragment comprises the amino acid substitution that differentiates the mutant protein from the wild type protein and making hybridoma cell lines that each produce a specific monoclonal antibody. The antibodies secreted by the different clones are then assayed for their ability to bind to the antigen using, e.g., ELISA or Antigen Microarray Assay, or immuno-dot blot technique. To detect the antibodies that are most specific for the detection of the protein of interest can be selected using routine methods and using the antigen and other antigens as well as positive controls comprising the wild type or mutant protein controls. The antibody that most specifically detects the desired antigen and protein and not other antigens or proteins will be selected for the detection assays.

The best clones can then be grown indefinitely in a suitable cell culture medium. They can also be injected into mice (in the peritoneal cavity, surrounding the gut) where they produce tumors secreting an antibody-rich ascites fluid from which the antibodies can be isolated and purified.

The antibodies can be purified using techniques that are well known to one of ordinary skill in the art.

In the methods and assays of the invention, the presence of any one or any combination of the mutant and wild type proteins is determined using antibodies specific for said proteins and detecting immunospecific binding of each antibody to its respective cognate marker.

Any suitable immunoassay method may be utilized, including those which are commercially available, to determine the level of each at least one of the specific proteins measured according to the invention. Extensive discussion of the known immunoassay techniques is not required here since these are known to those of skill in the art. Typical suitable immunoassay techniques include sandwich enzyme-linked immunoassays (ELISA), radioimmunoassays (RIA), competitive binding assays, homogeneous assays, heterogeneous assays, etc. Various of the known immunoassay methods are reviewed, e.g., in Methods in Enzymology, 70, pp. 30-70 and 166-198 (1980).

In the assays of the invention, “sandwich-type” assay formats can be used. These typically involve mixing the test sample with detection probes conjugated with a specific binding member (e.g., antibody) for the analyte (e.g., the urine sample) to form complexes between the analyte and the conjugated probes. These complexes are then allowed to contact a receptive material (e.g., antibodies) immobilized within the detection zone. Binding occurs between the analyte/probe conjugate complexes and the immobilized receptive material, thereby localizing “sandwich” complexes that are detectable to indicate the presence of the analyte. This technique may be used to obtain quantitative or semi-quantitative results. Some examples of such sandwich-type assays are described in by U.S. Pat. No. 4,168,146 to Grubb, et al. and U.S. Pat. No. 4,366,241 to Tom, et al. An alternative technique is the “competitive-type” assay. In a competitive assay, the labeled probe is generally conjugated with a molecule that is identical to, or an analog of, the analyte. Thus, the labeled probe competes with the analyte of interest for the available receptive material. Competitive assays are typically used for detection of analytes such as haptens, each hapten being monovalent and capable of binding only one antibody molecule. Examples of competitive immunoassay devices are described in U.S. Pat. No. 4,235,601 to Deutsch, et al., U.S. Pat. No. 4,442,204 to Liotta, and U.S. Pat. No. 5,208,535 to Buechler, et al.

The antibodies can be labeled. In some embodiments, the detection antibody is labeled by covalently linking to an enzyme, label with a fluorescent compound or metal, label with a chemiluminescent compound. For example, the detection antibody can be labeled with catalase and the conversion uses a colorimetric substrate composition comprises potassium iodide, hydrogen peroxide and sodium thiosulphate; the enzyme can be alcohol dehydrogenase and the conversion uses a colorimetric substrate composition comprises an alcohol, a pH indicator and a pH buffer, wherein the pH indicator is neutral red and the pH buffer is glycine-sodium hydroxide; the enzyme can also be hypoxanthine oxidase and the conversion uses a colorimetric substrate composition comprises xanthine, a tetrazolium salt and 4,5-dihydroxy-1,3-benzene disulphonic acid. In one embodiment, the detection antibody is labeled by covalently linking to an enzyme, label with a fluorescent compound or metal, or label with a chemiluminescent compound.

Direct and indirect labels can be used in immunoassays. A direct label can be defined as an entity, which in its natural state, is visible either to the naked eye or with the aid of an optical filter and/or applied stimulation, e.g., ultraviolet light, to promote fluorescence. Examples of colored labels which can be used include metallic sol particles, gold sol particles, dye sol particles, dyed latex particles or dyes encapsulated in liposomes. Other direct labels include radionuclides and fluorescent or luminescent moieties. Indirect labels such as enzymes can also be used according to the invention. Various enzymes are known for use as labels such as, for example, alkaline phosphatase, horseradish peroxidase, lysozyme, glucose-6-phosphate dehydrogenase, lactate dehydrogenase and urease. For a detailed discussion of enzymes in immunoassays see Engvall, Enzyme Immunoassay ELISA and EMIT, Methods of Enzymology, 70, 419-439 (1980).

In some embodiments, the immunoassay method or assay comprises a double antibody technique for measuring the level of the mutant and/or wild type proteins in the patient's body fluid, such as urine. According to this method one of the antibodies is a “capture” antibody and the other is a “detector” antibody. The capture antibody is immobilized on a solid support which may be any of various types which are known in the art such as, for example, microtiter plate wells, beads, tubes and porous materials such as nylon, glass fibers and other polymeric materials. In this method, a solid support, e.g., microtiter plate wells, coated with a capture antibody, preferably monoclonal, raised against the particular mutant and/or wild type protein of interest, constitutes the solid phase. Patient body fluid, e.g., urine, which may be diluted or not, typically at least 1, 2, 3, 4, 5, 10, or more standards and controls are added to separate solid supports and incubated. When the mutant protein is present in the body fluid it is captured by the immobilized antibody which is specific for the mutant protein in question. After incubation and washing, an anti-marker protein detector antibody, e.g., a polyclonal rabbit anti-marker protein antibody, is added to the solid support. The detector antibody binds to marker protein bound to the capture antibody to form a sandwich structure. After incubation and washing an anti-IgG antibody, e.g., a polyclonal goat anti-rabbit IgG antibody, labeled with an enzyme such as horseradish peroxidase (HRP) is added to the solid support. After incubation and washing a substrate for the enzyme is added to the solid support followed by incubation and the addition of an acid solution to stop the enzymatic reaction.

The degree of enzymatic activity of immobilized enzyme is determined by measuring the optical density of the oxidized enzymatic product on the solid support at the appropriate wavelength, e.g., 450 nm for HRP. The absorbance at the wavelength is proportional to the amount of S. Typhi protein in the fluid sample. A set of marker protein standards is used to prepare a standard curve of absorbance vs. e.g., mutant protein concentration. This method is useful because test results can be provided in 45 to 50 minutes and the method is both sensitive over the concentration range of interest for each mutant protein and is highly specific.

The antibody can be attached to a surface. Examples of useful surfaces on which the antibody can be attached for the purposes of detecting the desired antigen include nitrocellulose, PVDF, polystyrene, and nylon. The surface or support may also be a porous support (see., e.g., U.S. Pat. No. 7,939,342).

The standards may be positive samples comprising various concentrations of the at least one mutant protein to be detected to ensure that the reagents and conditions work properly for each assay. The standards also typically include a negative control, e.g., for detection of contaminants. In some aspects of the embodiments of the invention, the positive mutant and wild type controls may be titrated to different concentrations, including non-detectable amounts and clearly detectable amounts, and in some aspects, also including a sample that shows a signal at the threshold level of detection in the biological sample.

The assays can be carried out in various assay device formats including those described in U.S. Pat. Nos. 4,906,439; 5,051,237 and 5,147,609 to PB Diagnostic Systems, Inc.

The diagnosis of typhoid fever can be made if the presence of any one of the mutant proteins is detected in the patient's sample, such as a blood or urine sample.

In addition to presence of the mutant protein in the sample, one can also measure the quantity of the mutant protein in the sample using routine methods known to one skilled in the art.

The assay devices used according to the invention can be arranged to provide a quantitative or a qualitative (present/not present) result.

The assays may be carried out in various formats including, as discussed previously, a microtiter plate or a microfluidic device format are particularly useful for carrying out the assays in a batch mode. The assays may also be carried out in automated immunoassay analyzers which are well known in the art and which can carry out assays on a number of different samples. These automated analyzers include continuous/random access types. Examples of such systems are described in U.S. Pat. Nos. 5,207,987 and 5,518,688 to PB Diagnostic Systems, Inc. Various automated analyzers that are commercially available include the OPUS® and OPUS MAGNUM® analyzers.

Another assay format which can be used according to the invention is a rapid manual test which can be administered at the point-of-care at any location. Typically, such point-of-care assay devices will provide a result which is either “positive” i.e. showing the protein is present, or “negative” showing that the protein is absent. Typically, a control showing that the reagents worked in general is included with such point-of-care system. Point-of-care systems, assays and devices have been well described for other purposes, such as pregnancy detection (see, e.g., U.S. Pat. No. 7,569,397; U.S. Pat. No. 7,959,875).

### Nucleic Acid Primers and Probes

Nucleic acid primers and probes may be designed for the amplification of the target nucleic acids around the mutations described herein using the nucleic acid sequences provided herein. The primers and probes may be of any convenient length varying from about 10-25, 15-20, 15-15, 10-30 bases long primers to array probes varying from 10 bp up to 1000 bp long.

The probes and primers may be labeled for the detection. One can also label the nucleic acid amplification products, such as the allele-specific amplification products using DNA dyes.

Useful labels include, but are not limited to, intercalating dyes, such as ethidium bromide and propidium iodide, minor-groove binders, such as DAPI and the Hoechst dyes, and other nucleic acid stains, including acridine orange, 7-AAD, LDS 751 and hydroxystilbamidine. In addition, fluorescent labels, such as, TOTO, TO-PRO and SYTOX families of dyes, as well as SYTO family of dyes, and Amine-reactive SYBR dye can be used. While not preferred, also radioactive labels can naturally be used, and include, e.g., S³⁵ or P³².

Chemical modifications produce shifts in the absorption and emission spectra and reduce the quantum yields of the bound dyes but cause little or no change in their high affinity for DNA. The names of the dyes reflect their basic structure and spectral characteristics. For example, YOYO-1 iodide (491/509) has one carbon atom bridging the aromatic rings of the oxacyanine dye and exhibits absorption/emission maxima of 491/509 nm when bound to dsDNA. YOYO-3 dye (612/631)-which differs from YOYO-1 dye only in the number of bridging carbon atoms—has absorption/emission maxima of 612/631 nm when bound to dsDNA. Fluorescence spectra for the POPO, BOBO, YOYO, TOTO, JOJO and LOLO dyes are described in Molecular Probes®, Molecular Probes Handbook, A Guide to Fluorescent Probes and Labeling Technologies, 11th Edition, Iain Johnson (Editor), Michelle T. Z. Spence (Editor).

Because of its high sensitivity, fluorescence is useful for nucleic acid analysis. Prior to carrying out the experiment, the sample is labeled by means of a suitable fluorochrome.

If the nucleic acid detection method uses a microarray, binding is typically achieved in a separate incubation step and the final result is obtained after appropriately washing and drying of the micro-array. Micro-array readers usually acquire information about the fluorescence intensity at a given time of the binding process that would ideally be the time after arriving at the thermodynamic equilibrium.

Alternatively, the mutations can be detected on a DNA array, chip or a microarray. In such an embodiment, probes that are specific for mutant and/or normal alleles can be affixed to surfaces for use as “gene chips.”

Such gene or mutation-specific probe-comprising chips are included as one embodiment of this invention and they can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the present invention also can be used for fluorescent detection of the mutant sequences. Such techniques have been described, for example, in U.S. Pat. Nos. 5,968,740 and 5,858,659. A probe also can be affixed to an electrode surface for the electrochemical detection of nucleic acid sequences such as described by Kayyem et al. U.S. Pat. No. 5,952,172 and by Kelley, S. O. et al. (1999) Nucleic Acids Res. 27:4830-4837.

Oligonucleotides corresponding to the mutant and/or wild-type allele are immobilized on a chip which is then hybridized with labeled nucleic acids of a test sample obtained from a patient. A positive hybridization signal is obtained with a sample containing the mutation and/or wild-type sequence. In a homozygous sample only the mutation comprising sequence shows a signal, in a heterozygous sample, both the mutant and the wild-type alleles are detected and in the wild-type allele containing samples only the wild-type allele is detected.

Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

A microarray is an array of discrete regions, typically nucleic acids, which are separate from one another and are typically arrayed at a density of between, about 100/cm² to 1000/cm2, but can be arrayed at greater densities such as 10000/cm². The principle of a microarray experiment, is that the alleles amplified from the nucleic acid sample are labeled, e.g., during amplification, are used to generate a labeled sample, termed the ‘target’, which is hybridized in parallel to a large number of, nucleic acid sequences, typically single-stranded DNA sequences, immobilized on a solid surface in an ordered array.

In one embodiment, the invention provides mutation detection arrays for the diagnosis of autism spectrum disorder and/or intellectual disability.

The arrays provided in the invention comprise at least one of the novel detected mutations, in some embodiments two, three, four, five, six or more of the mutations disclosed herein are represented as probes on the arrays. Wild type alleles may or may not be present on the same array. In some embodiments all the mutant alleles as well as their wild type equivalents are represented by at least one probe on an array.

Any number of different probes ranging from one to tens of thousands of nucleic acid species can be detected simultaneously using microarrays. Although many different microarray systems have been developed the most commonly used systems today can be divided into two groups, according to the arrayed material: complementary DNA (cDNA) and oligonucleotide microarrays. The arrayed material has generally been termed the probe since it is equivalent to the probe used in a northern blot analysis. Probes for cDNA arrays are usually PCR products generated from cDNA libraries or clone collections, using either vector-specific or gene-specific primers, and are printed onto glass slides or nylon membranes as spots at defined locations. Spots are typically 10-300 m in size and are spaced about the same distance apart. Using this technique, arrays consisting of more than 30,000 cDNAs can be fitted onto the surface of a conventional microscope slide. For oligonucleotide arrays, short 20-25 mers are synthesized in situ, either by photolithography onto silicon wafers (high-density-oligonucleotide arrays from Affymetrix or by ink-jet technology (developed by Rosetta Inpharmatics, and licensed to Agilent Technologies).

Alternatively, presynthesized oligonucleotides can be printed onto glass slides. Methods based on synthetic oligonucleotides offer the advantage that because sequence information alone is sufficient to generate the DNA to be arrayed, no time-consuming handling of cDNA resources is required. Also, probes can be designed to represent the most unique part of a given transcript, making the detection of closely related genes or splice variants possible. Although short oligonucleotides may result in less specific hybridization and reduced sensitivity, the arraying of presynthesized longer oligonucleotides (50-100 mers) has been developed to counteract these disadvantages.

The Affymetrix HG-U133.Plus 2.0 gene chips can be used and hybridized, washed and scanned according to the standard Affymetrix protocols. Some nucleic acid probes can be replicated on arrays or different probes detecting the same mutation can be included as controls, making 96 the total number of available hybridizations for subsequent analysis.

Although the same procedures and hardware described by Affymetrix could be employed in connection with the present invention, other alternatives are also available. Many reviews have been written detailing methods for making microarrays and for carrying out assays (see, e.g., Bowtell, Nature Genetics Suppl. 27:25-32 (1999); Constantine, et al, Life ScL News 7:11-13 (1998); Ramsay, Nature Biotechnol. 16:40-44 (1998)). In addition, patents have issued describing techniques for producing microarray plates, slides and related instruments (U.S. Pat. No. 6,902,702; U.S. Pat. No. 6,594,432; U.S. Pat. No. 5,622,826, which are incorporated herein in their entirety by reference) and for carrying out assays (U.S. Pat. No. 6,902,900; U.S. Pat. No. 6,759,197 which are incorporated herein in their entirety by reference). The two main techniques for making plates or slides involve either polylithographic methods (see U.S. Pat. No. 5,445,934; U.S. Pat. No. 5,744,305 which are incorporated herein in their entirety by reference) or robotic spotting methods (U.S. Pat. No. 5,807,522 which are incorporated herein in their entirety by reference). Other procedures may involve inkjet printing or capillary spotting (see, e.g., WO 98/29736 or WO 00/01859 which are incorporated herein in their entirety by reference).

The substrate used for microarray plates or slides can be any material capable of binding to and immobilizing oligonucleotides including plastic, metals such a platinum and glass. One substrate is glass coated with a material that promotes oligonucleotide binding such as polylysine (see Chena, et al, Science 270:467-470 (1995)). Many schemes for covalently attaching oligonucleotides have been described and are suitable for use in connection with the present invention (see, e.g., U.S. Pat. No. 6,594,432 which is incorporated herein in its entirety by reference). The immobilized oligonucleotides should be, at a minimum, 20 bases in length and should have a sequence exactly corresponding to a segment in the gene targeted for hybridization.

In some embodiments, apparatus and related methods are used to obtain the sample, for example, machines described in U.S. Pat. No. 4,120,448, U.S. Pat. No. 5,879,280 and U.S. Pat. No. 7,241,281, which are incorporated herein in their entirety by reference.

The invention further provides microfluidic devices for the detection of the mutant alleles causing autism spectrum disorders and/or intellectual disability. The components of the assays, namely, nucleic acid probes that hybridize to the mutant and/or wild-type alleles of the genes disclosed herein and the reagents needed for detection of the hybridized nucleic acids from a biological sample comprising nucleic acids from the individual, fetus or pre-implantation embryo described herein can be used in the format of a microfluidic device. Such devices have been well described in the art, see, e.g., U.S. Pat. Nos. 6,444,461; 6,479,299; 7,041,509, incorporated herein by reference in their entirety.

The microfluidic devices can be designed to comprise a channel or chamber that contains one or more probes, such as nucleic acid probes against one or more of the mutant and/or wild type alleles, or antibodies specific for the mutant or the wild type protein preferably immobilized on the channel or chamber surface. The device can be supplied with appropriate buffers for binding the nucleic acids or proteins from a sample, such as a blood sample to the antibodies and detecting the bound proteins either inside the device or eluting them out and detecting them in the eluted sample.

The methods and assays can be performed using one probe or primer pair per reaction. The methods and assays may also be performed in multiplex format that can detect at least two mutant alleles in one reaction, multiplexing, e.g., 1-10, 2-5, 2-6, 2-10, 2, 3, 4, 5, 6, 7, 8, 9, 10, 10-15, and 10-20 reactions are contemplated. In such multiplex analyses, other mutations that are known to cause autism spectrum disorders can be added.

### Histone Modifying Drugs

Various histone modifying drugs have been developed and are currently either in clinical trials or in use. The following is a list of such therapeutic agents that can be used for treatment of autism spectrum disorders and/or intellectual disability: Vorinostat (SAHA, ZOLINZA®) (FDA approved); Belinostat; LAQ824; Panobinostat; Pyroxamide; Givinostat; PCI-24781; Romidepsin; AN 9; Sodium Phenylbutyrate; Valproic acid sold as BACECA®, SAVICOL™, or AVUGANE; Entinostat; Tacedinaline; MGCD 0103; DACOGEN™; VIDAZA® (FDA approved); Anacardic acid; Curcumin; Isothiazolones; Garcinol; MB-3; H₃-CoA-20; AMI-1; AMI-5; Stilbamidine; and DZNep.

For example, valproic acid sold also as BACECA®, SAVICOL™, or AVUGANE, is an anticonvulsant and it is used to control absence seizures, tonic-clonic seizures (grand mal), complex partial seizures, juvenile myoclonic epilepsy and the seizures associated with Lennox-Gastaut syndrome. It is also used in treatment of myoclonus. In some countries, parenteral (administered intravenously) preparations of valproate are used also as second-line treatment of status epilepticus, as an alternative to phenyloin. Valproate is one of the most common drugs used to treat post-traumatic epilepsy. Valproic acid is also FDA approved for the treatment of manic episodes associated with bipolar disorder, adjunctive therapy in multiple seizure types (including epilepsy), and prophylaxis of migraine headaches. It is more recently being used to treat neuropathic pain.

VIDAZA (Azacitidine) is currently used to treat myelodysplastic syndrome (a group of conditions in which the bone marrow produces blood cells that are misshapen and does not produce enough healthy blood cells). Azacitidine is in a class of medications called demethylation agents.

ZOLINZA (Vorinostat) is used to treat cutaneous T-cell lymphoma (CTCL, a type of cancer) in people whose disease has not improved, has gotten worse, or has come back after taking other medications. Vorinostat is in a class of medications called histone deacetylase (HDAC) inhibitors.

Belinostat (PXD 101) sold by Spectrum Pharmaceuticals, is a novel HDAC inhibitor in late stage clinical development with more than 700+ patients treated to date. Belinostat has shown to be well tolerated which would allow for combination with traditional chemotherapy without causing further bone marrow toxicity. In pre-clinical trials belinostat has shown to be effective against multiple cancers.

### Administration of Drugs

The drugs or pharmaceutical agents can be administered using any convenient or effective route, and preferably systemically, although also local, such as intracranial administration is contemplated. Systemic administration can be oral, intravenous administration, parenteral administration, subcutaneous administration or intramuscular. While the oral administration may be the most convenient, the agents can also be administered using nebulizers in inhalers or through subcutaneous patches with sustained release formula.

The dosages can be easily optimized using routine clinical practices and the knowledge of the dosages in which the drugs indicated above have been used for other indications. The patients' age, weight, gender, and other conditions, including responsiveness and possible side effects will be taken into account when determining the proper dosages.

Effectiveness of the treatment in the case of autism or autism spectrum disorders or intellectual disability can be measured by observing any positive change in the clinical symptoms of the patient.

### Computer Systems and Automated Mutation Analysis

The methods of the invention can be automated using robotics and computer directed non-human systems. The biological sample comprising nucleic acids or proteins can be injected into a system, such as a microfluidic devise entirely run by a robotic station from sample input to output of the result. The term “computer” as it is referred to herein indicates a non-human machine, not a human brain.

The step of displaying the result can also be automated and connected to the same system or in a remote system. Thus, the sample analysis can be performed in one location and the comparison and the result analysis in another location, the only connection being, e.g., an internet connection in such way that the analysis result can be fed from the analysis module to the comparison module which can then either in the same location or by sending the result to a third location, which may or may not be the same location as the first location wherein the analysis was performed, to be displayed in a format suitable for either reading by a health professional or by a patient.

In one embodiment, the analysis, comparison and the result is performed in one location. In some embodiments, the analysis is performed in one location and the comparison and the displaying the results are performed at a different location.

The invention also contemplates computer readable media that comprises information on the status of the detected alleles in the genes discloses. For example, the information may include the step of analysis whether or not the mutation in the HIST3H3 gene is homozygous or heterozygous in the nucleic acid or protein sample. The information may also include information regarding whether or not any of the CLDC mutations is present in a compound heterozygous form in a sample.

Another aspect of the invention provides a computer readable and executable program product (i.e., software product) for use in a computer device that executes program instructions recorded in a computer-readable medium to perform calculations relating to the presence and/or absence of alleles in the sample and whether they are heterozygous, homozygous, compound heterozygous or trans heterozygous with respect to any other mutation that may be included on the array or microfluidic device other detection system in the biological sample comprising nucleic acids or proteins, such as blood sample from a human subject, plasma sample from a pregnant mother or a cell sample from a pre-implantation embryo.

In one embodiment, the program product comprises: a recordable medium and a plurality of computer-readable instructions executable by the computer device to analyze data obtained from a method used to determine the alleles as disclosed herein, to transmit such expression level information one location to another (e.g., from the apparatus used for the gene expression measurements to the computer, or alternatively, the data can be inputted into the computer from a recordable medium, e.g., CD-ROM, USB drives etc). Computer readable media include, but are not limited to, CD-ROM disks (CD-R, CD-RW), DVD-RAM disks, DVD-RW disks, floppy disks and magnetic tape.

It will be apparent to those of ordinary skill in the art that methods involved in the present invention may be embodied in a computer program product that includes a computer usable and/or readable medium. For example, such a computer usable medium may consist of a read only memory device, such as a CD ROM disk or conventional ROM devices, or a random access memory, such as a hard drive device or a computer diskette, having a computer readable program code stored thereon.

### Prenatal Diagnostics

Prenatal diagnosis or prenatal screening is testing for diseases or conditions in a fetus or embryo before it is born.

Diagnostic prenatal testing can be by invasive or non-invasive methods. An invasive method involves probes or needles being inserted into the uterus, e.g. amniocentesis, which can be done from about 14 weeks gestation, and usually up to about 20 weeks, and chorionic villus sampling, which can be done earlier (between 9.5 and 12.5 weeks gestation) but which may be slightly more risky to the fetus. However since chorionic villus sampling is performed earlier in the pregnancy than amniocentesis, typically during the first trimester, it can reasonably be expected that there will be a higher rate of miscarriage after chorionic villus sampling than after amniocentesis.

Prenatal diagnostics can also be performed using a nucleic acid sample obtained, isolated or enriched, e.g., from maternal plasma, or chorionic villus using methods that are well known to one skilled in the art. For example, fetal nucleic acids have been generally found to represent only about 3-6% of the nucleic acids circulating in the maternal blood (Lo et al, Am J Hum Genet 62, 768-775, 1998). Thus the prenatal diagnostic methods of the present invention can be performed from a nucleic acid sample taken from the mother, such as maternal plasma. In some embodiments, one can enrich the fetal nucleic acids to improve the analysis of the fetal nucleic acids from maternal plasma or blood samples. Methods to enrich fetal nucleic acids listed, e.g., in U.S. Pat. No. 7,785,798 can be used in prenatal diagnostic applications of the methods as described herein.

Pre-natal diagnostic methods provide options for parents to make reproductive decisions and to prepare for therapy options.

### Pre-Implantation Diagnostics

The methods and assays as disclosed herein are also useful in pre-implantation diagnostics. In such embodiments, fertilized embryos are screened for mutant alleles of HIST3H3, AMT, GLDC, and PEX7 genes and only embryos with wild type alleles are implanted in the uterus. Alternatively, one may also elect to implant embryos which are heterozygous for the HIST3H3 mutations R54C, R129C and/or R130C. In some embodiments, if the embryo carries more than one mutation in an allele or in the alleles for any one of the genes selected from HIST3H3, AMT, PEX7 and GLDC, and if wild type allele carrying embryos are available, one elects to discard the mutation carrying embryos as the phenotypic expression based on our results regarding the compound heterozygozity, e.g., in the GLDC gene, or transheterozygozity with other possible mutations would be unclear.

In medicine and (clinical) genetics pre-implantation genetic diagnosis (PGD or PIGD) (also known as embryo screening) refers to procedures that are performed on embryos prior to implantation, sometimes on oocytes prior to fertilization. PGD is considered another way to prenatal diagnosis. When used to screen for a specific genetic disease, its main advantage is that it avoids selective pregnancy termination as the method makes it highly likely that the baby will be free of the disease under consideration. PGD thus is an adjunct to assisted reproductive technology, and requires in vitro fertilization (IVF) to obtain oocytes or embryos for evaluation.

The term pre-implantation genetic screening (PGS) is used to denote procedures that do not look for a specific disease but use PGD techniques to identify embryos at risk. Although typically, in medicine, to “diagnose” means to identify an illness or determine its cause, in the preimplantation screening or diagnostic methods the embryo may technically not be ill. An oocyte or early-stage embryo has no symptoms of disease. Rather, they may have a genetic condition that could lead to disease. To “screen” means to test for anatomical, physiological, or genetic conditions in the absence of symptoms of disease. So both PGD and PGS should be referred to as types of embryo screening. The terms are used interchangeably in this application.

Procedures performed on sex cells before fertilization may instead be referred to as methods of oocyte selection or sperm selection, although the methods and aims partly overlap with PGD. Although not as preferably, the assays for detecting the mutations of the present invention may also be used from oocyte or sperm samples. In this method, if one of the oocytes or sperm to be used in the IVF only carries a wild type allele of the genes indicated herein, the sperm or oocyte may be used in the IFV with reduced risk of disease in the offspring.

PGD is available for a large number of monogenic disorders, that is, a condition is due to a single gene only, (autosomal recessive, autosomal dominant or X-linked disorders) or a chromosomal structural aberration (such as a balanced translocation). PGD helps these couples identify embryos carrying a genetic disease or a chromosome abnormality, thus avoiding diseased offspring. The most frequently diagnosed autosomal recessive disorders are cystic fibrosis, Beta-thalassemia, sickle cell disease and spinal muscular atrophy type 1. The most common dominant diseases are myotonic dystrophy, Huntington's disease and Charcot-Marie-Tooth disease; and in the case of the X-linked diseases, most of the cycles are performed for fragile X syndrome, haemophilia A and Duchenne muscular dystrophy. Though it is quite infrequent, some centers report PGD for mitochondrial disorders or two indications simultaneously.

As these methods are well established, same methodology may be used in connection with the application of the diagnostic assays of the present invention to detection of these mutations in a pre-implantation embryo.

In addition, there are infertile couples or same sex female couples who carry an inherited condition and who opt for PGD as it can be easily combined with their IVF treatment.

Currently, most of the PGD embryos are obtained by assisted reproductive technology. In order to obtain a large group of oocytes, the patients undergo controlled ovarian stimulation (COH). COH is carried out either in an agonist protocol, using gonadotrophin-releasing hormone (GnRH) analogues for pituitary desensitisation, combined with human menopausal gonadotrophins (hMG) or recombinant follicle stimulating hormone (FSH), or an antagonist protocol using recombinant FSH combined with a GnRH antagonist according to clinical assessment of the patient's profile (age, body mass index (BMI), endocrine parameters). hCG is administered when at least three follicles of more than 17 mm mean diameter are seen at transvaginal ultrasound scan. Transvaginal ultrasound-guided oocyte retrieval is scheduled 36 hours after hCG administration. Luteal phase supplementation consists of daily intravaginal administration of 600 μg of natural micronized progesterone.

Oocytes are denudated from the cumulus cells, as these cells can be a source of contamination during the PGD if PCR-based technology is used. In the majority of the reported cycles, intracytoplasmic sperm injection (ICSI) is used instead of IVF. The main reasons are to prevent contamination with residual sperm adhered to the zona pellucida and to avoid unexpected fertilization failure. The ICSI procedure is carried out on mature metaphase-II oocytes and fertilization is assessed 16-18 hours after. The embryo development is further evaluated every day prior to biopsy and until transfer to the woman's uterus. During the cleavage stage, embryo evaluation is performed daily on the basis of the number, size, cell-shape and fragmentation rate of the blastomeres. On day 4, embryos were scored in function of their degree of compaction and blastocysts were evaluated according to the quality of the throphectoderm and inner cell mass, and their degree of expansion.

As PGD can be performed on cells from different developmental stages, the biopsy procedures vary accordingly. Theoretically, the biopsy can be performed at all preimplantation stages, but only three have been suggested: on unfertilised and fertilised oocytes (for polar bodies, PBs), on day three cleavage-stage embryos (for blastomeres) and on blastocysts (for trophectoderm cells).

The biopsy procedure involves two steps: the opening of the zona pellucida and the removal of the cell(s). There are different approaches to both steps, including mechanical, chemical (Tyrode's acidic solution) and laser technology for the breaching of the zona pellucida, extrusion or aspiration for the removal of PBs and blastomeres, and herniation of the trophectoderm cells.

The first and second polar body of the oocyte are extruded at the time of the conclusion of the meiotic division, normally the first polar body is noted after ovulation, and the second polar body after fertilization. PB biopsy is used mainly by two PGD groups in the USA (Verlinsky Y, Ginsberg N, Lifchez A, Valle J, Moise J, Strom C M (October 1990). “Analysis of the first polar body: preconception genetic diagnosis”. Hum. Reprod. 5 (7): 826-9; Munné S, Dailey T, Sultan K M, Grifo J, Cohen J (April 1995). “The use of first polar bodies for preimplantation diagnosis of aneuploidy”. Hum. Reprod. 10 (4): 1014-20) and by groups in countries where cleavage-stage embryo selection is banned (Montag M, van der Ven K, Dorn C, van der Ven H (October 2004). “Outcome of laser-assisted polar body biopsy and aneuploidy testing”. Reprod. Biomed. Online 9 (4): 425-9). They have been used for diagnosing translocations and monogenic disorders of maternal origin, as well as for PGS.

The first PB is removed from the unfertilised oocyte, and the second PB from the zygote, shortly after fertilization. The main advantage of the use of PBs in PGD is that they are not necessary for successful fertilisation or normal embryonic development, thus ensuring no deleterious effect for the embryo. One of the disadvantages of PB biopsy is that it only provides information about the maternal contribution to the embryo, which is why cases of autosomal dominant and X-linked disorders that are maternally transmitted can be diagnosed, and autosomal recessive disorders can only partially be diagnosed. Another drawback is the increased risk of diagnostic error, for instance due to the degradation of the genetic material or events of recombination that lead to heterozygous first PBs. It is generally agreed that it is best to analyse both PBs in order to minimize the risk of misdiagnosis. This can be achieved by sequential biopsy, necessary if monogenic diseases are diagnosed, to be able to differentiate the first from the second PB, or simultaneous biopsy if FISH is to be performed.

For example, in Germany, where the legislation bans the selection of preimplantation embryos, PB analysis is the only possible method to perform PGD. The biopsy and analysis of the first and second PBs can be completed before syngamy, which is the moment from which the zygote is considered an embryo and becomes protected by the law.

Cleavage-stage biopsy is generally performed the morning of day three post-fertilization, when normally developing embryos reach the eight-cell stage. The biopsy is usually performed on embryos with less than 50% of anucleated fragments and at an 8-cell or later stage of development. A hole is made in the zona pellucida and one or two blastomeres containing a nucleus are gently aspirated or extruded through the opening. The main advantage of cleavage-stage biopsy over PB analysis is that the genetic input of both parents can be studied. On the other hand, cleavage-stage embryos are found to have a high rate of chromosomal mosaicism, putting into question whether the results obtained on one or two blastomeres will be representative for the rest of the embryo. It is for this reason that some programs utilize a combination of PB biopsy and blastomere biopsy. Furthermore, cleavage-stage biopsy, as in the case of PB biopsy, yields a very limited amount of tissue for diagnosis, necessitating the development of single-cell PCR and FISH techniques. Although theoretically PB biopsy and blastocyst biopsy are less harmful than cleavage-stage biopsy, this is still the prevalent method. It is used in approximately 94% of the PGD cycles reported to the ESHRE PGD Consortium. The main reasons are that it allows for a safer and more complete diagnosis than PB biopsy and still leaves enough time to finish the diagnosis before the embryos must be replaced in the patient's uterus, unlike blastocyst biopsy. Of all cleavage-stages, it is generally agreed that the optimal moment for biopsy is at the eight-cell stage. It is diagnostically safer than the PB biopsy and, unlike blastocyst biopsy, it allows for the diagnosis of the embryos before day 5. In this stage, the cells are still totipotent and the embryos are not yet compacting. Although it has been shown that up to a quarter of a human embryo can be removed without disrupting its development, it still remains to be studied whether the biopsy of one or two cells correlates with the ability of the embryo to further develop, implant and grow into a full term pregnancy.

In an attempt to overcome the difficulties related to single-cell techniques, it has been suggested to biopsy embryos at the blastocyst stage, providing a larger amount of starting material for diagnosis. It has been shown that if more than two cells are present in the same sample tube, the main technical problems of single-cell PCR or FISH would virtually disappear. On the other hand, as in the case of cleavage-stage biopsy, the chromosomal differences between the inner cell mass and the trophectoderm (TE) can reduce the accuracy of diagnosis, although this mosaicism has been reported to be lower than in cleavage-stage embryos.

TE biopsy has been shown to be successful in animal models such as rabbits (Gardner R L, Edwards R G (April 1968). “Control of the sex ratio at full term in the rabbit by transferring sexed blastocysts”. Nature 218 (5139): 346-9) mice (Carson S A, Gentry W L, Smith A L, Buster J E (August 1993). “Trophectoderm microbiopsy in murine blastocysts: comparison of four methods”. J. Assist. Reprod. Genet. 10 (6): 427-33) and primates (Summers P M, Campbell J M, Miller M W (April 1988). “Normal in-vivo development of marmoset monkey embryos after trophectoderm biopsy”. Hum. Reprod. 3 (3): 389-93). These studies show that the removal of some TE cells is not detrimental to the further in vivo development of the embryo.

Human blastocyst-stage biopsy for PGD is performed by making a hole in the ZP on day three of in vitro culture. This allows the developing TE to protrude after blastulation, facilitating the biopsy. On day five post-fertilization, approximately five cells are excised from the TE using a glass needle or laser energy, leaving the embryo largely intact and without loss of inner cell mass. After diagnosis, the embryos can be replaced during the same cycle, or cryopreserved and transferred in a subsequent cycle.

There are two drawbacks to this approach, due to the stage at which it is performed. First, only approximately half of the preimplantation embryos reach the blastocyst stage. This can restrict the number of blastocysts available for biopsy, limiting in some cases the success of the PGD. Mc Arthur and coworkers (McArthur S J, Leigh D, Marshall J T, de Boer K A, Jansen R P (December 2005). “Pregnancies and live births after trophectoderm biopsy and preimplantation genetic testing of human blastocysts”. Fertil. Steril. 84 (6): 1628-36) report that 21% of the started PGD cycles had no embryo suitable for TE biopsy. This figure is approximately four times higher than the average presented by the ESHRE PGD consortium data, where PB and cleavage-stage biopsy are the predominant reported methods. On the other hand, delaying the biopsy to this late stage of development limits the time to perform the genetic diagnosis, making it difficult to redo a second round of PCR or to rehybridize FISH probes before the embryos should be transferred back to the patient.

Sampling of cumulus cells can be performed in addition to a sampling of polar bodies or cells from the embryo. Because of the molecular interactions between cumulus cells and the oocyte, gene expression profiling of cumulus cells can be performed to estimate oocyte quality and the efficiency of an ovarian hyperstimulation protocol, and may indirectly predict aneuploidy, embryo development and pregnancy outcomes (Fauser, B. C. J. M.; Diedrich, K.; Bouchard, P.; Dominguez, F.; Matzuk, M.; Franks, S.; Hamamah, S.; Simon, C. et al. (2011). “Contemporary genetic technologies and female reproduction”. Human Reproduction Update 17 (6): 829-8470; Demko Z, Rabinowitz M, Johnson D (2010). “Current Methods for Preimplantation Genetic Diagnosis”. Journal of Clinical Embryology 13 (1): 6-12).

### Kits

Embodiments of the invention as described herein also provide for the design and preparation of assays, and kits comprising detection reagents needed to the identify the allelic variants of the genes identified herein in a biological sample comprising nucleic acids or proteins. Particularly, the detection reagents are designed and prepared to identify the mutations in the genes as identified throughout this specification. Examples of detection reagents that can be used to identify the mutations in the genes identified here in a test sample can include a primer and a probe, wherein the probe can selectively hybridize to at least one mutant and/or wild-type allele of the gene or an antibody that selectively recognizes the mutant and/or wild type protein. Primers for amplification, reverse transcription from an mRNA sample and for mutation detection can be provided.

Also provided are reagents and kits thereof for practicing one or more of the above described methods. The subject reagents and kits thereof may vary greatly. Reagents of interest include reagents specifically designed for use in detection of HIST3H3, AMT, PEX7, and GLDC gene mutations as disclosed herein.

In some embodiments of the kits, specifically mutations HIST3H3 gene, AMT gene, GLDC gene or PEX7 gene. In the case of a kit comprising nucleic acids, the kit comprises at least one probe, and in some embodiments several probes to detect at least one mutation in the HIST3H3 gene that results in an amino acid change in the critical R-residues of the HIST3H3 gene, e.g., a substitution R54H, R129C, or R130C; at least one probe to detect at least one mutation in the AMT gene that results in E211K substitution in the AMT protein; at least one probe to determine if a sample comprises a compound heterozygous mutation resulting in any one of the amino acid change combinations of L90F/V705M, L90F/G18C, or A569T/A97V in a GLDC protein; and/or at least one probe to determine whether the sample comprises or a heterozygous mutation resulting in an amino acid change W75C in a PEX7 protein or a heterozygous amino acid change I308F in the AMT protein.

In another embodiment, the kit comprises at least one antibody that binds with differentiating specificity to any of the above-identified mutant proteins when compared to a wild-type protein so that the antibody can be used to determine the presence of absence of the mutant protein in a patient sample. In some embodiments, the kit comprises two antibodies, one of which is specific for the wild type protein and the other is specific for the mutant protein. These two antibodies in combination can be used to determine if the biological sample comprises a homozygous or a heterozygous change in the respective gene. In a homozygous case, only the antibody with affinity to one of the alleles will bind, in a heterozygous case, both antibodies provide a signal. In kits designed for a compound heterozygous sample, one antibody can be specific to detect both mutations, but typically, two antibodies both specific to the specific mutant alleles are included.

The kits can include at least one reagent specific for detecting for one or more mutations described herein, and optionally instructions for using the reagents and for determining the presence or absence of the mutant and/or the wild-type allele in the biological sample. The kit may include containers, such as vials with or without appropriate reagents or buffers. The kit may also include reagents and components for obtaining the biological sample, such as a blood sample.

The kits of the subject invention may include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g., hybridization and washing buffers, prefabricated probe arrays, labeled probe purification reagents and components, like spin columns, etc., signal generation and detection reagents, e.g., streptavidin-alkaline phosphatase conjugate, chemifluorescent or chemiluminescent substrate, and the like.

In addition to the above components, the kits may further include instructions for practicing the methods and arrays described herein. These instructions may be present in the kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site.

A related aspect of the invention provides kits comprising the program products described herein. The kits may also optionally contain paper and/or computer-readable format instructions and/or information, such as, but not limited to, information on protein or nucleic acid microarrays, on tutorials, on experimental procedures, on reagents, on related products, on available experimental data, on using kits, on agents for treating inflammatory diseases, including their toxicity, and on other information. The kits optionally also contain in paper and/or computer-readable format information on minimum hardware requirements and instructions for running and/or installing the software.

The following examples provide discussion regarding the methods used in the discovery of the specific genes and mutations and their associations with autism spectrum disorders and/or intellectual disability. They are not intended exclusive, but exemplary and the embodiments of the invention, such as assays, methods, arrays and kits are based on the discoveries described herein below.

### EXAMPLES

### Identification of Novel Mutations in the Autism Spectrum Diseases

### Materials and Methods

Quantitative real-time RT-PCR: Total RNA from human fetal and adult brain was purchased from BioChain Institute Inc. (Hayward, Calif.) and OriGene Technologies, Inc. (Rockville, Md.), respectively. cDNA was synthesized from 1 μg of RNA using the SUPERSCRIPT® III First-Strand Synthesis System (Invitrogen Corporation, Carlsbad, Calif.). Quantitative real-time PCR reactions were performed on 100 ng of cDNA using TAQMAN® Gene Expression master mix (Applied Biosystems, Carlsbad, Calif.) and commercially available primers and probes (Applied Biosystems, Carlsbad, Calif.). All RNA samples were analyzed in triplicate and normalized relative to Gapdh levels. The quantitative real-time PCR data were analyzed using the ΔΔCt method.

Subcloning and mutagenesis: Full-length human cDNAs were obtained (HIST3H3-EGFP, NM_{—}003493.2, GeneCopoeia; PEX7, NM_{—}000288) and used for subcloning and site-directed mutagenesis. QUIKCHANG® Lightning Site-Directed Mutagenesis Kit (Agilent Technologies, Inc., Santa Clara, Calif.) was used to introduce R53H, R128C, or R129C into HIST3H3-EGFP and W75C into PEX7. The cDNA for PEX7 was subcloned into the BstBI and NheI sites in the mammalian expression vector pReceiver-M03 (GeneCopoeia, Rockville, Md.), which also introduced a C-terminal EGFP tag.

Transfection and immunostaining: HeLa cells were transfected, using LIPOFECTAMINE 2000 according to the manufacture's protocol (Invitrogen Corporation, Carlsbad, Calif.), with pReceiver-HIST3H3, pReceiver-HIST3H3-R53H, pReceiver-HIST3H3-R128C, or pReceiver-HIST3H3-R129C constructs (150 ng each). After 24 hours, cells were fixed in 4% paraformaldehyde-PBS for 15 minutes at room temperature. After washing with 1×PBS, cells were blocked in 1×PBS containing 5% goat serum and 0.1% Triton-X (blocking buffer) for 1 hour at room temperature. Cells were then incubated in primary antibody diluted in blocking buffer overnight at 4° C. The primary antibodies used were: chicken anti-GFP (Abcam, 1:1000), rabbit anti-CENP-A (Cell Signaling, 1:400), and rabbit anti-acetyl-Histone H4 (Millipore, 1:100). Cells were then washed with 1×PBS three times and incubated in secondary antibody diluted in blocking buffer (ALEXA FLUOR® 488 goat anti-chicken IgG, 1:400; ALEXA FLUOR® 555 goat anti-rabbit IgG, 1:400; Invitrogen) for 1 hour at room temperature. After washing with 1×PBS for three times, cells were mounted with SLOWFADE® Gold antifade reagent with DAPI (Invitrogen Corporation, Carlsbad, Calif.). All experiments were performed in duplicate.

Since autism is known to be extremely genetically heterogeneous, with multiple different genetic syndromes causing indistinguishable phenotypes, we developed a strategy to sort this heterogeneity. To enrich for recessive mutations and to provide genetic power to identify single point mutations, we ascertained families in which children with autism spectrum disorder (ASD) were born to parents who were related (consanguineous), typically as cousins.

### Results

We recruited >200 families and phenotyped them initially using diagnostic criteria according to the Diagnostic and Statistical Manual of Psychiatric Disease IV-Revised (DSMIV-R), as well as additional quantitative instruments). We performed more extensive analysis of selected families that were most informative genetically, using consanguinity to identify candidate genetic syndromes that could be analyzed in larger numbers of patients with ASD. For each family we performed genome-wide linkage analysis using high-throughput single nucleotide polymorphism (SNP) arrays, and performed exclusionary mapping, reasoning that a proportion of families would show homozygous recessive mutations, and that these recessive mutations would lie within larger blocks of homozygosity. Typically, single offspring of first cousin parents showed ≈11% of their genome as homozygous (almost double the theoretical prediction of 6.25% due typically to the presence of additional loops of consanguinity besides the index first cousin marriage), whereas two affected offspring of cousin parents shared homozygosity for ≈1% of the genome and three affected offspring shared homozygosity for ≈0.2% of the genome. We performed high-throughput DNA sequencing of these regions using DNA capture with custom Nimblegen arrays, or performed whole exome sequencing, focusing analysis on those rare DNA changes that were homozygous.

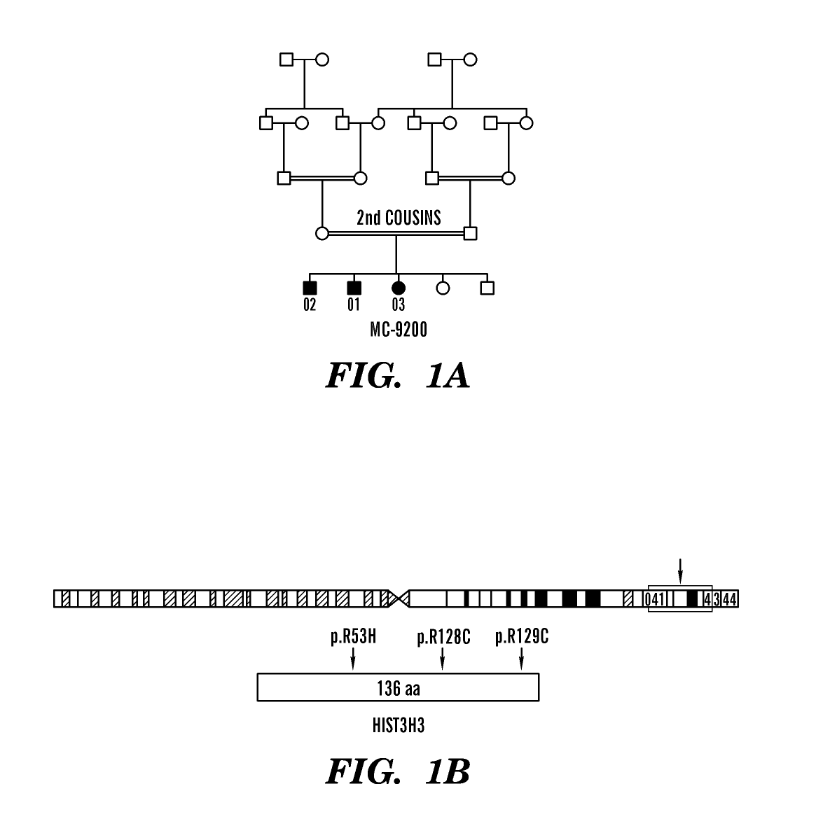

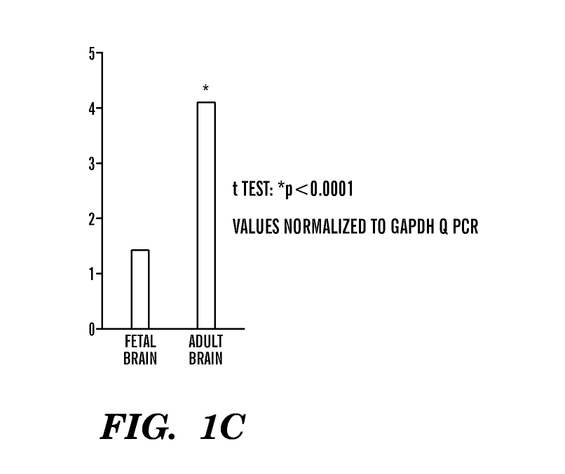

We ascertained a family from Pakistan with three children affected with intellectual disability (ID) and autistic features (FIG. 1A). Their parents were second cousins, and all affected offspring showed shared homozygosity over a 23 Mb region on distal chromosome 1q41-43 (LOD 2.8) as well as a smaller 4 Mb region at chr1p32 (FIG. 1B). Array capture and high throughput sequencing of all UCSC-annotated exons and flanking 50 bp from this interval yielded a total of 433 variants. Of these, all but 28 were present in dbSNP130 or the 1000 Genomes project. Of the remaining novel variants, only four were predicted to be potentially deleterious, i.e., non-synonymous, splice-site disrupting, or frame altering, and therefore candidate mutations.

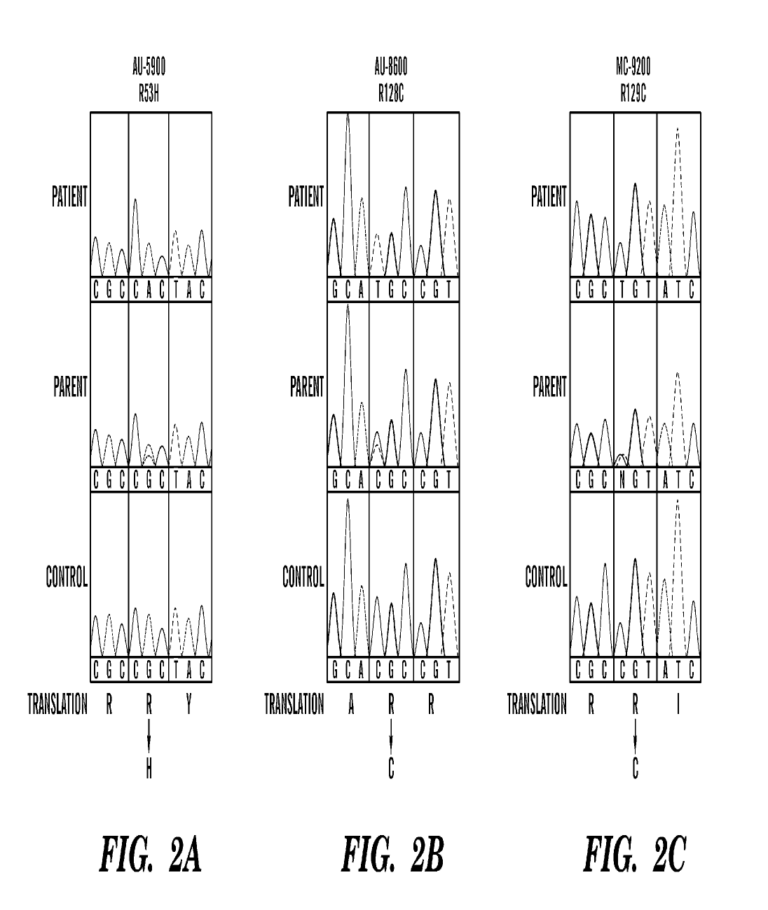

Two of these were ruled out on the basis of carrier frequency in controls, and a third candidate (C1orf168) is an uncharacterized open reading frame (ORF). The remaining candidate gene, HIST3H3, encodes a histone H3 protein (Histone 3.1t), and bore a homozygous c.388 C>T substitution (chr1:226679262 G>A, hg18) that creates an R129C (or R130C with respect to SEQ ID NO: 1) substitution (FIG. 2C), altering an amino acid that is conserved in all histone H3 proteins down to yeast. This mutation is absent from 532 control individuals (1064 chromosomes) and is homozygous in all three affected children, and heterozygous in parents and unaffected siblings. Further analysis of HIST3H3 in 24 other families with intellectual disability or autism spectrum disorder (ASD) identified mutations in two additional ASD families. An ASD proband from a consanguineous simplex Turkish family (AU-8600) showed linkage (LOD<1.5) and a mutation that results in an R128C (FIG. 2B), a change that is also absent from 532 normal individuals. A third consanguineous simplex family (AU-5900) with a child affected with ASD showed an R53H HIST3H3 mutation (FIG. 2A), which was absent in the homozygous state from 532 normal individuals, although 1/532 normal individuals carried this allele in the heterozygous state, consistent with the carrier state or status of a very rare recessive disease. R53H and R128C were each homozygous in the single affected individual from each family, and heterozygous in parents and unaffected siblings (FIG. 1A).

All three of these arginine residues are highly conserved between species in histone H3 proteins, and have been shown to be potential sites of histone methylation. R129 may be especially critical in modulating interactions between histone H3 proteins and ASF1A (FIG. 1C, English et al. Structural basis for the histone chaperone activity of Asf (Cell (2006) vol. 127 (3): 495-508; Natsume et al. Structure and function of the histone chaperone CIA/ASF1 complexed with histones H3 and H4. Nature (2007) vol. 446 (7133) pp. 338), which mediates H3K56 acetylation (Das et al. CBP/p300-mediated acetylation of histone H3 on lysine 56. (Nature (2009) Vol. 459 (7243): 113-7).

Sequence analysis of the entire HIST3H3 gene in 531 ASD probands from the Autism Genetic Resource Exchange (AGRE) collection, as well as parallel analysis of 532 controls, did not show any other homozygous (or compound heterozygous) mutations of HIST3H3 in other ASD cases (or controls), and showed very few heterozygous variants (3 in 532 controls, and 5 in 521 ASD cases) suggesting variants of any kind in this gene are quite rare, and are not a common cause of ASD in outbred families, but are presumably slightly enriched in consanguineous populations.

The central importance of histone H3's to synaptic function and plasticity has been recently noted (Ma et al Nature Neuroscience 2010, Borrelli et al Neuron 2008). RT-PCR analysis of HIST3H3 confirmed that it is expressed in the human brain, at higher levels in adult brain than in developing brain (FIG. 1C). To understand the potential implications of the HIST3H3 mutations found in these three families, we analyzed the localization of EGFP-tagged HIST3H3 in cultured HeLa cells. Overexpressed HIST3H3 robustly localizes to the nucleus, where it appears to be enriched in large, globular regions of heterochromatin, demonstrated by exclusion from domains of immunoreactivity to anti-acetylated histone H4, a marker of euchromatin. Mutant HIST3H3 constructs (R53H, R128C, and R129C) retained nuclear localization, but demonstrated an altered pattern of staining.

AU-1700 represents a Saudi family with multiple children affected by autism which provided evidence for a different genetic mechanism by which recessive mutation can lead to autism. This family had three children who were affected with autism spectrum disorders and seizures (FIG. 2A), and exhibited homozygosity for a single region on 3p22-14, encompassing 18 Mb and >300 genes. Array-capture and high-throughput sequence analysis of this region revealed 856 homozygous, rare variants, of which 100 were novel, and 8 altered protein coding regions.

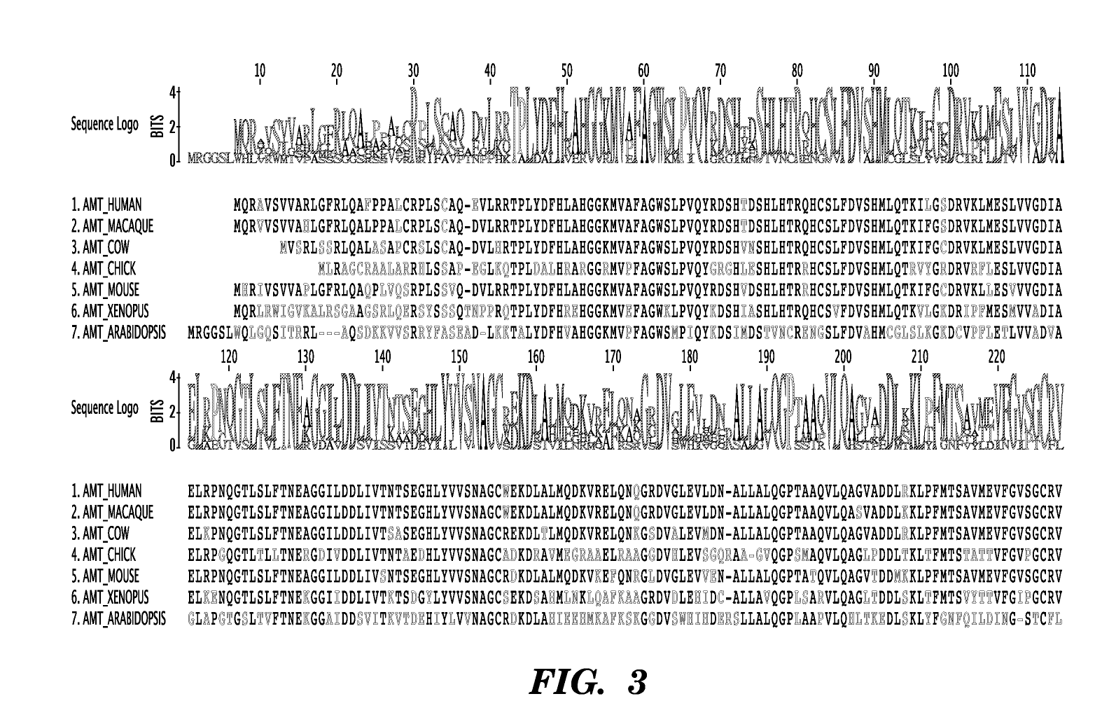

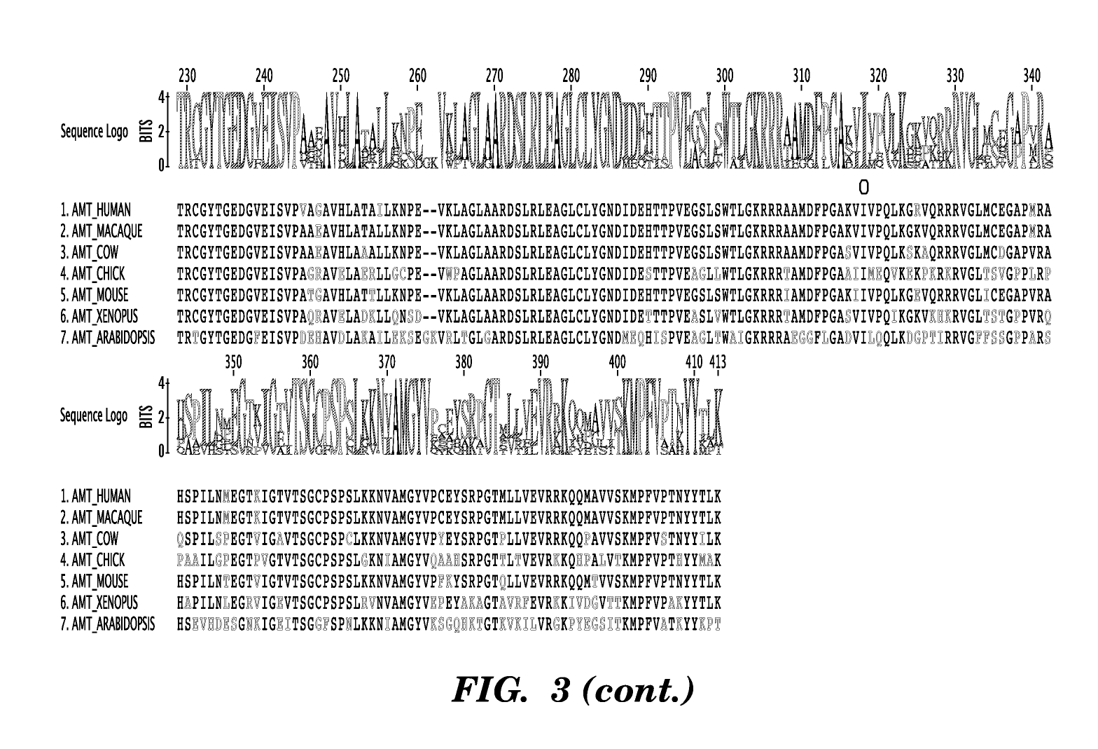

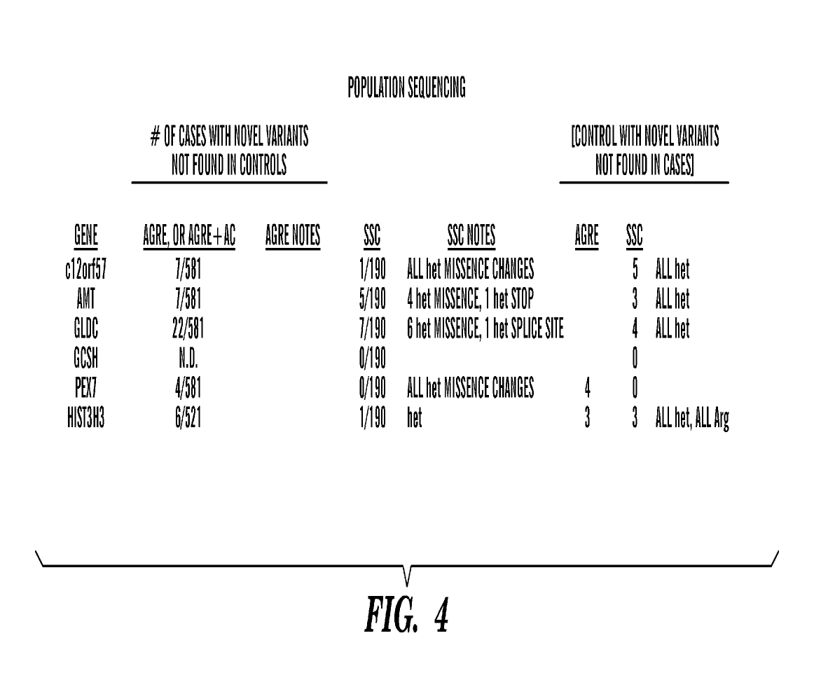

Genotyping of these SNPs in normal individuals revealed four of these to be common polymorphisms, and review of allele frequencies, amino acid conservation, and disease association of the four remaining genes (NBEAL2, WDR6, USP4, and AMT) pointed to AMT as the most likely causative gene. This mutation encodes an I308F missense change that was absent in 510 Sanger-sequenced normal individuals. Isoleucine 308 resides in domain 3 of the AMT protein, a domain important for capping domains 1 (important for folding) and domain 2 (containing catalytic residues). This residue is conserved in all AMT sequences in all species down to mosquito (FIG. 3) and based on the AMT crystal structure it resides in a buried hydrophobic pocket. Mutation of isoleucine to a bulkier phenylalanine group would be predicted to disrupt this pocket.

Mutations in AMT are known to cause a familiar, Mendelian syndrome called nonketotic hyperglycinemia (NKH, also known as glycine encephalopathy) (Applegarth and Toone. Glycine encephalopathy (nonketotic hyperglycinaemia): review and update. J Inherit Metab Dis, 2004, vol. 27(3): 417-22) characterized by neonatal lethargy, intractable seizures, and death. In retrospect, symptoms in the three children, if they had all co-occurred in a single child, would have strongly suggested the diagnosis of mild NKH: since one child had transient coma, and all children had seizures as well as language delay and abnormal socialization. Prominent autistic symptoms have been described before in mild cases of NKH, caused by hypomorphic mutations, and the mildest reported cases of NKH also lack the deteriorating course typically seen in NKH, suggesting that these autistic children also show a very mild case of NKH due to a hypomorphic mutation.

Classical NKH is caused by mutations in the glycine cleavage system, a highly conserved metabolic pathway consisting of GLDC, AMT, and GCSH. Classical NKH is associated with GLDC mutation ˜85% of the time, with most of the remaining cases accounted for by AMT. Generally, two inactivating mutations (one maternal and one paternal) are the rule. However, in 25% of cases associated with GLDC mutation, only a single inactivating mutation is found.

Since rare, “private” copy number variation (CNV) is another established cause of autism spectrum disorders, we screened a database of clinical chromosomal microarray results in a cohort of patients with autism and found two patients with deletions at the GLDC locus predicted to remove the first seventeen exons. Results of copy number analysis of the Simons Simplex Collection (SSC), a large database of carefully phenotyped children with higher functioning autism, revealed a third patient with a de novo CNV in the GCSH gene. None of these CNV's were ever seen in normal individuals, suggesting that spontaneous copy number variants at the NKH loci are a potential cause of autism.

To explore the potential relevance of NKH mutations in a broader sample of ASD patients, we analyzed the two major NKH genes, AMT and GLDC, in American patients diagnosed with ASD from several sources. First, we sequenced AMT and GLDC in 771 autism patients (519 AGRE patients, 190 SSC patients, and 62 Autism Consortium patients) and filtered for common variation present in dbSNP130. We found four patients with homozygous or compound inactivating mutations in AMT or GLDC: two patients with homozygous mutations in AMT (E211K), and two patients with compound heterozygous mutations in the GLDC gene (L90F/V705M and G18C, A569T/A97V). E211K was previously reported as a GLDC “helper” mutation that increased the severity of another pathogenic change (R320H). L90F and A97V alter highly conserved residues in GLDC, and both V705M and A569T represent mutations previously reported in patients with classical NKH. G18C of indeterminate pathogenicity. E211K, V705M, and A569T, were found at a low frequency in controls (1.4%, 0.3%, and 0.7%, respectively), but never in combination with other (non-dbSNP) variants, consistent with their pathogenicity. In addition, two ASD patients were found to bear heterozygous splice site GLDC mutations predicted to cause protein truncation.

Sequence analysis of AMT and GLDC in large numbers of cases and controls suggests an important role for simultaneous heterozygous mutation of both genes in autism. Sequence analysis was performed using Sanger sequencing in 584 cases and 510 controls for AMT, GLDC, and GCSH. These three proteins form a complex to catalyze the metabolism of glycine into CO₂, CH₃, 5,10-methylene-tetra-hydrofolate, and reduced pyridine. GLDC is a glycine decarboxylase, AMT is a methyl transferase, and GCSH is a hydrogen carrier protein. Variants were screened against dbSNP130 and the 1000 Genomes project to rule out common polymorphisms. Overall rates of heterozygous mutations in any of these three genes were nominally, but not significantly, greater in cases versus controls. Next we explored the hypothesis that compound heterozygous mutations (i.e., two different deleterious mutations in the two alleles of the same gene) or transheterozygous mutations (i.e., one deleterious mutation in one gene, and one deleterious mutation in another, unlinked gene, but which encode proteins that function together) are another disease-causing mechanism for this pathway. Overall, two simultaneous heterozygous mutations were significantly more common in affected patients than in controls (p<0.02, Fisher exact test, two-tailed).

However, two mutations in the same gene were not substantially more common in cases than in controls, though phase analysis was not available to determine in each patient whether the two mutations were on the same chromosome (in which case only one mutation would be deleterious) or on different chromosomes (in which case the gene would be inactivated by the two mutations). In order to focus on the most specific mutations, we examined alleles that were already known to be causative of NKH by having been previously identified in NKH patients (through the Human Gene Mutation Database, HGMD). This resulted in 10 cases potentially compound heterozygous for two NKH mutations, whereas only 2 controls were potential compound heterozygotes (p<0.04, Fisher two-tailed test). Strikingly however, 7 cases (1.3%) showed a known, disease-associated heterozygous mutation in AMT, as well as a heterozygous known mutation in GLDC, whereas no controls showed these two non-linked transheterozygous mutations (p<0.01, Fisher two-tailed). Re-analysis of transheterozygotes suggested that xx cases showed either known disease-associated mutations or rare, predicted deleterious mutations in AMT as well as GLDC, whereas xx controls showed two known or predicted mutations, suggesting that transheterozygous mutations in glycine pathway genes alone account for at least 1% of autism in this sample.

A third family (AU-3500) with three affected children showed linkage to a 10.5 Mb region of chromosome 6 (LOD>2.4), and array capture analysis suggested another hypomorphic recessive mutation. The linked region contained genes, and sequence analysis revealed 321 homozygous potentially damaging mutations, of which only 2 were not present in additional controls genotyped. One of these remaining potential mutations is a W75C mutation in PEX7, the receptor required for the import of PTS2-containing proteins into the peroxisome (Braverman et al, Hum Mutat. 2002 October; 20(4):284-97). PEX7 when completely null causes rhizomelic chondrodysplasia punctatum (RCDP), a syndrome of abnormal facies, cataracts, skeletal dysplasia, and severe psychomotor defects. The W75C mutation lies in a structural WD-40 repeat of PEX7 and disrupts a tryptophan residue conserved in species down to yeast, and was absent from >500 normal controls. Furthermore, PEX7 W75C fails to complement the peroxisomal targeting defect in a PEX7-null human cell line, further confirming it as a mutation, although presumably hypomorphic since it is not associated with the full spectrum of RCDP in this family. In fact, specific mutations consistent with the formation of some residual PEX7 protein have been described that cause a milder syndrome of intellectual dysfunction with none of the dysmorphic stigmata of RCDP, and a child previously reported with partial loss-of-function in PEX7 was a determined to be autistic (Braverman et al, Hum Mutat. 2002 October; 20(4):284-97). Re-sequencing of PEX7 in 581 autism cases revealed four patients with missense mutations in conserved residues and predicted to be damaging that were absent from controls, although none had clear compound heterozygous mutations.

Analysis of three other families further supported additional roles for specific mutations causing autism, whereas other mutations in the same gene show more severe phenotypes.

Though most American families sampled by the AGRE collection are of mixed European ancestry and share no known near ancestors in common, a small proportion of European-American parents will either share a traceable common ancestor, or may share common ethnic ancestry for both parents, which in either case may result in homozygosity for recessive mutations, as has been demonstrated for a host of known Mendelian recessive diseases. We analyzed the AGRE collection and identified “outlier” families, in which the affected children show degrees of homozygosity far higher than would be expected from parents with no common ancestry. We performed whole exome sequencing in 18 of these patients, reasoning that a fraction of the runs of homozygosity would contain homozygous causative mutations. We then analyzed the whole exome sequence to identify rare, likely deleterious changes. We obtained an average coverage of 92% at 20×, and identified ˜34,581 variants per exome. Common variants identified by the 1000 Genomes project and dbSNP130 were filtered out, and the remaining variants were subject to an in-house bioinformatic pipeline to annotate variants that may disrupt gene function (by altering the coding sequence or truncating the protein). On average, 736 variants per exome were potentially pathogenic, and out of these, 39 were homozygous changes. Using an independent technology, we genotyped these candidate variants in the 18 probands as well as their family members, allowing us to examine segregation within the family and also segregation with disease since a lot of these families had multiple affected individuals as well as unaffected siblings.

Starting with an average of 39 homozygous variants per exome, we were able to successfully validate 33% of the variants, which is pretty good considering that for novel heterozygous changes, 95% of variants turn out to be cell line artifacts. A smaller subset of these variants fell within runs of homozygosity, allowing us to narrow down the number of candidate variants to an average of 4 variants per exome, and for four families only one variant segregated with the disease. The data are summarized in Table 2. For some families our approach did not yield any candidate variants, which is expected since homozygous variants will not necessarily be causative in all the families studied. We found that there is a rich burden of potentially deleterious variation in the autism exome, and after validation and checking segregation, we were able to narrow it down to very few candidate genes per family. These were all novel genes, and as examples, they were involved in small GTPase mediated signal transduction, transcriptional regulation, protein modification processes, and RNA splicing (Table 3). Different patients showed candidate mutations in different genes, suggesting that recessive autism genes are heterogeneous. Our data also suggests that autistic children may have mutations in novel genes not previously associated with disease.

Here we show that recessive mutations are important causes of autism. Homozygous null mutations appear to be an exceedingly rare cause of ASD. On the other hand, linked, homozygous missense changes were found in three genes (AMT, PEX7, HIST3H3) in four families with ASD. In the case of PEX7 and AMT, it is known that null mutations of these same genes cause a much more severe Mendelian phenotype in which autistic symptoms are an occasional feature. These missense mutations appear to be consistent with hypomorphic mutations that seem to cause a much milder phenotype associated with prominent ASD. NKH mutations, especially transheterozygous ones, alone appear to be involved in >1% of cases of autism in the AGRE collection. Whereas we have not studied other potential candidate genes to determine the broad importance of such trans-heterozygous mutations, many metabolic disorders cause autistic symptoms at some point in their course, to say nothing of potential non-allelic complementation of the synaptic pathways. Multiple, heterogeneous, metabolic disorders underlying some cases of autism may also potentially explain the anecdotal response of some autistic children to unusual dietary changes.

Transheterozygous mutations in two unlinked genes with related biochemical function may be a very important mechanism in autism and other milder cognitive disorders (e.g., attention deficit hyperactive disorder (ADHD), dyslexia, mild intellectual disability (ID)). Transheterozygous recessive mutations are presumably important in cancer, and have been implicated in lipid disorders, but may have broader applicability to the analysis of complex disease.

Transheterozygous mutations in animal models are almost invariably milder than homozygous mutations, and so it would be expected that they would not cause the same Mendelian disorder caused by two mutations in the same gene. The critical variable to identifying them is likely to be combining genomic data on mutations with proteomic data on biochemical pathways, since transheterozygous mutations typically occur in genes that encode proteins that physically interact.

Two major models exist to explain nonallelic noncomplementation (NANC): the dosage model and the poisson model. In the former, NANC affects proteins whose function is unusually dosage sensitive, whereas in the poison model, particular alleles, typically missense alleles and not nonsense mutations, have slight “dominant negative” function that is not recognized in the homozygous state, but is revealed in the transheterozygous state. To the extent that the poison model ultimately holds for NKH mutations, it may be possible to annotate specific AMT variants that are likely to show transheterozygous noncomplementation. In humans, transheterozygous mutations may ultimately form a horizon between Mendelian genetics and the “common disease, multiple rare alleles” hypothesis in which more than two heterozygous mutations may interact further. However, genes with known Mendelian phenotypes may form a guide to identify candidates that can be then tested in combination for mutations in complex diseases.

Our results illustrate the importance and the challenges of whole exome sequencing in an extremely heterogeneous condition such as autism. Each exome contains large numbers of variants that initially seem to defy analysis. Almost all instances in which new genetic syndromes have been identified using whole exome or whole genome sequencing have involved families with recessive disorders generally (Miller syndrome) and/or shared parental ancestry specifically (Gardner syndrome; WDR62), because the analysis of homozygous mutations provides tremendous power to improve “signal to noise” caused by sequencing errors, spontaneous cell line mutations, somatic mutations, etc. Hence, tracing ancestry may be an important tool to define genetic causes in a subset of patients.

Our data also identify several unexpected and potentially remediable pathways in ASD. Glycine metabolism has not been previously implicated, but fits well with theories of autism that involve mismatches of excitatory/inhibitory balance in the nervous system. Histone biology has been studied in relation to cancer, and several agents that regulate histone biology are in trials for cancer and other disorders, but may present an unexpected avenue towards autism treatment as well. Defects in histone biology also would complement the potential importance of activity-regulated gene expression in autism, since histone modifications are crucial regulators of short term, and long-term, gene expression in the brain.

1. An in vitro assay comprising a step of analyzing a biological sample from a human individual for at least one mutation in HIST3H3 gene, AMT gene, GLDC gene or PEX7 gene, wherein a homozygous nucleic acid mutation resulting in an amino acid mutation selected from R54H, R129C, or R130C in a HIST3T3 protein or E211K in a AMT protein; a compound heterozygous mutation resulting in any one of the amino acid mutation combinations of L90F/V705M, L90F/G18C, or A569T/A97V in a GLDC protein; or a heterozygous mutation resulting in an amino acid mutation W75C in a PEX7 protein or a heterozygous amino acid mutation I308F in the AMT protein indicates that the autism spectrum disorder and/or intellectual disability in the individual is caused by the identified mutation or mutations.

2. The in vitro assay of claim 1, further comprising a step of determining whether or not a histone modulating agent is useful as an optional treatment for the individual, wherein the presence of the mutation in the HIST3H3 gene that results in a homozygous mutation R54H, R129C, or R130C in the HIST3H3 protein indicates that histone modulating agents are useful as an optional treatment for the individual, and wherein the absence of the mutation in the HIST3H3 gene that results in a homozygous mutation R54H, R129C, or R130C in the HIST3H3 protein indicates that the histone modulating agents are not useful as an optional treatment for the individual.

3. The in vitro assay of claim 1, wherein prior to the step of determining the individual has been assessed by a clinical evaluation and considered as having clinical symptoms of autism spectrum disorder and/or intellectual disability.

4. The in vitro assay of claim 1, wherein the step of analyzing comprises contacting the biological sample with at least one probe which forms a complex with its target nucleic acid or protein and is therefore capable of detecting at least one of the nucleic acid mutations or amino acid mutations.

5. The in vitro assay of claim 4, wherein the probe is a nucleic acid.

6. The in vitro assay of claim 4, wherein the probe is an antibody.

7. The in vitro assay of claim 1, wherein the step of analyzing comprises a step of nucleic acid amplification and/or nucleic acid sequencing.

8. The in vitro assay of claim 6, wherein the assay is an immunoassay.

9. The in vitro assay of claim 1, wherein the step of analyzing comprises a computer implemented analysis of one or more sequences, wherein the analysis comprises comparing sequence information from the biological sample to a reference and/or displaying the result of a comparison.

10. An in vitro assay for prenatal diagnosis of a fetus or pre-implantation diagnosis of an embryo for autism spectrum disorder and/or intellectual disability comprising analyzing a biological sample comprising fetal or pre-implantation embryonic nucleic acids for a mutation in HIST3H3 gene, AMT gene, GLDC gene or PEX7 gene, wherein a homozygous mutation resulting in an amino acid change of any one of R54H, R129C, and R130C in a HIST3T3 protein and E211K in a AMT protein; a compound heterozygous mutation resulting in an amino acid change combination of any one of L90F/V705M, L90F/G18C, and A569T/A97V in a GLDC gene; or an amino acid change of W75C in a PEX7 protein or I308F in the AMT protein is indicative that the fetus or the pre-implantation embryo is affected with autism spectrum disorder and/or intellectual disability.

11. The in vitro assay of claim 10, wherein the step of analyzing comprises nucleic acid sequencing of the HIST3H3 gene, AMT gene, GLDC gene or PEX7 gene or a portion of said genes.

12. The in vitro assay of claim 10, wherein the step of analyzing comprises contacting the fetal nucleic acid with at least one probe capable of hybridizing to one or more of the mutant forms of the HIST3H3 gene, AMT gene, GLDC gene and/or PEX7 gene.

13. The in vitro assay of claim 12, wherein the probe is attached to a solid surface.

14. The in vitro assay of claim 10, wherein the step of analyzing comprises a computer readable medium that allows automatic, computerized, non-human performed comparison of information from the nucleic acid sample with a reference and/or an automatic display of the identified mutations if any.

15. The in vitro assay of claim 10 further comprising the step of implanting the embryo if the embryo is a homozygous for the wild type allele R54, R129, and R130 in a HIST3T3 protein and E211 in a AMT protein; a L90/V705, L90/G18, and A569/A97 in a GLDC gene; or W75 in a PEX7.

16. An in vitro assay for determining an optional therapeutic intervention for an individual for the treatment of autism spectrum disorder and/or intellectual disability comprising the steps of analyzing a biological sample obtained from the individual by contacting the biological sample with at least one probe capable of detecting a nucleic acid mutation resulting in R54H, R129C, or R130C amino acid mutation in HIST3H3 gene, wherein if the mutation is detected and is homozygous, the individual is determined as a candidate for an optional therapeutic intervention with a histone modulating agent.

17. A method of treating autism spectrum disorder and/or intellectual disability comprising the steps of (a) determining if the individual is homozygous for a mutation in the HIST3H3 gene resulting in a homozygous amino acid change R54H, R129C, or R130C in the HIST3H3 protein; and (b) administering a histone modulating agent to the individual if the individual is homozygous for a mutation in the HIST3H3 gene resulting in an amino acid change R54H, R129C, or R130C in the HIST3H3 protein.

18. A nucleic acid array comprising at least one probe to detect at least one mutation or a pair of mutations selected from a HIST3H3 gene resulting in an amino acid change R54H, R129C, or R130C in a HIST3H3 protein, a mutation in an AMT gene resulting in an amino acid change E211K in an AMT protein; mutations in an GLDC gene resulting in a pair of amino acid changed L90F/V705M, L90F/G18C, or A569T/A97V in an GLDC protein; and a mutation in a PEX7 gene resulting in an amino acid change W75C in a PEX7 protein.

19. A kit for the diagnosis of autism spectrum disorder and/or intellectual disability comprising at least one probe to detect at least one mutation or a pair of mutations selected from a HIST3H3 gene resulting in an amino acid change R54H, R129C, or R130C in a HIST3H3 protein, a mutation in an AMT gene resulting in an amino acid change E211K in an AMT protein; a mutation in an GLDC gene resulting in a pair of amino acid changed L90F/V705M, L90F/G18C, or A569T/A97V in an GLDC protein; and a mutation in a PEX7 gene resulting in an amino acid change W75C in a PEX7 protein for the diagnosis of autism spectrum disorder and/or intellectual disability.

20. The kit of claim 19, wherein the probe is attached to a solid surface.

21-22. (canceled)
